SEQUENCE LISTING (6 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 42 aa, Sus scrofa
YAEGTFISDYSIAMDKIRQQDFVNWLLAQKGKKSDWKHNITQ

SEQ ID NO: 3 - protein, 42 aa, Bos taurus
YAEGTFISDYSIAMDKIRQQDFVNWLLAQKGKKSDWIHNITQ

SEQ ID NO: 4 - protein, 42 aa, Mesocricetus auratus
YAEGTFISDYSIAMDKIRQQDFVNWLLAQKGKKNDWKHNITQ

SEQ ID NO: 5 - protein, 42 aa, Rattus norvegicus
YAEGTFISDYSIAMDKIRQQDFVNWLLAQKGKKNDWKHNLTQ

SEQ ID NO: 6 - protein, 42 aa, Mus musculus
YAEGTFISDYSIAMDKIRQQDFVNWLLAQRGKKSDWKHNITQ

(19) United States
(12) Patent Application Publication
(10) Pub. No.: US 2009/0286722 A1
(43) Pub. Date: Nov. 19, 2009

(54) Analogs of Gastric Inhibitory Polypeptide as a Treatment for Age Related Decreased Pancreatic Beta Cell Function

(75) Inventors: Peter Raymond Flatt, County Antrim (GB); Finbarr Paul Mary O'Harte, County Londonderry (GB)

Correspondence Address: K&L Gates LLP, STATE STREET FINANCIAL CENTER, One Lincoln Street, BOSTON, MA, 02111-2950, US

(73) Assignee: Utech Limited, Coleraine, County Londonderry, NI (GB)

(21) Appl. No.: 11/991,638

(22) PCT Filed: Sep. 8, 2006
(86) PCT No.: PCT/EP06/08778

§ 371 Date: Feb. 6, 2009

Related U.S. Application Data
(60) Provisional application No. 60/715,334, filed on Sep. 8, 2005.
(60) Provisional application No. 60/715,335, filed on Sep. 8, 2005.

(51) Int. Cl. A61K 38/16; C07K 7/08; C07K 14/00; A61K 38/10; A61P 1/18

(52) U.S. Cl. 514/12; 530/327; 530/326; 530/325; 530/324; 514/14; 514/13

(57) ABSTRACT

Peptide analogues and methods are provided for treating age-related symptoms of decreased pancreatic beta-cell function, including glucose intolerance, type 2 diabetes, beta-cell glucose insensitivity, insulin resistance and reduced insulin secretion.

### FIELD OF THE INVENTION

The present invention relates to use of peptide analogues of glucose-dependent insulinotropic polypeptide (gastric inhibitory polypeptide; GIP) for the manufacture of medicaments for ameliorating or restoring age related decreased pancreatic beta-cell function. The present invention also relates to certain novel analogues and pharmaceutical compositions containing them.

### BACKGROUND

In humans and other mammals, the ability to regulate serum glucose and insulin levels changes with age. Increasing age is associated with a deterioration of glucose tolerance and insulin secretion, with a defective early insulin response to glucose (Elahi, D. et al., 1985, Endocrinology 116:11-16). In adult humans and many other mammalian species, glucose-stimulated plasma insulin concentrations have been shown to decrease with age (Bailey, C. J., and Flatt, P. R., 1982, Int. J. Obesity 6:11-21). For example, an age-related deterioration of glucose-mediated insulin release was reported in Wistar rats accompanied by a progressive decline of insulin messenger RNA and total pancreatic insulin content (Perfetti, R. et al., 1995, Am. J. Physiol. 269:E983-990). In addition to deterioration of glucose tolerance and insulin secretion, aging is accompanied by a progressive decline of insulin sensitivity (Chang, A. M., and Halter, J. B., 2003, Am. J. Physiol. Endocrinol. Metab. 284:E7-12; Defronzo, R. A., 1979, Diabetes 28:1095-1101).

### SUMMARY OF THE INVENTION

Methods and uses are provided for treating age-related symptoms of decreased pancreatic beta-cell function. As used herein, “age-related symptoms of decreased pancreatic beta-cell function” is the deterioration in pancreatic beta-cell function and/or mass that accompanies increasing age. The term “age-related symptoms of decreased pancreatic beta-cell function” includes changes in glucose tolerance, beta-cell glucose sensitivity, beta cell mass, insulin resistance and reduced insulin secretion that occur in otherwise healthy individuals and excludes those individuals with diagnosed or undiagnosed prediabetes or type 1 diabetes or type 2 diabetes. Peptide analogues of gastric inhibitory polypeptide, for treating such symptoms and other symptoms of type 1 and type 2 diabetes, as well as prediabetes, are also provided, as well as pharmaceutical compositions containing the peptide analogues.

The invention includes a method of ameliorating or restoring age related decreased pancreatic beta-cell function, including treating glucose intolerance, loss of beta cell mass, beta-cell glucose insensitivity and/or insulin resistance in a mammal, where the method includes administering to a mammal a therapeutically effective amount of a pharmaceutical composition comprising a peptide analogue of GIP, where the peptide analogue potentiates glucose-induced insulin secretion. The invention also includes a method of ameliorating or restoring age related decreased pancreatic beta-cell function, including treating glucose intolerance, loss of beta cell mass, beta-cell glucose insensitivity and/or insulin resistance in a mammal, where the method includes administering to a mammal a therapeutically effective amount of a pharmaceutical composition comprising a peptide analogue of at least 12 amino acid residues from the N-terminal end of GIP(1-42).

The symptoms and conditions to be treated are due to advancing age.

In these methods, the peptide analogue can include modification in the form of N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule.

Any of the peptide analogues used in the methods described herein can be covalently attached to a polyethylene glycol (PEG) molecule.

The pharmaceutical compositions used in the methods can include a pharmaceutically acceptable carrier. The peptide analogue can be in the form of a pharmaceutically acceptable salt, such as a pharmaceutically acceptable acid addition salt. The pharmaceutical compositions can also include an agent having an antidiabetic effect.

The peptide analogues used in the methods can include a modification by fatty acid addition at an epsilon amino group of at least one lysine residue, the modification comprising the reaction of an acyl radical having a saturated or unsaturated, linear or branched aliphatic chain, of from 4 to 22 carbons.

Heteroatoms and cyclic hydrophobic groups can be tolerated so long as the aliphatic nature of the acyl radical is not significantly disturbed. Suitable acyl radicals include, but are not limited to, a C-8 octanoyl group, C-10 decanoyl group, C-12 lauroyl group, C-14 myristoyl group, C-16 palmitoyl group, C-18 stearoyl group, or C-20 acyl group (each of which may be substituted so long as the aliphatic nature of the acyl radical is not disturbed) to the epsilon amino group of a lysine residue, for instance, the linking of a C-16 palmitoyl group to a lysine residue chosen from the group consisting of Lys¹⁶, Lys³⁰, Lys³², Lys³³ and Lys³⁷.

The peptide analogue can be N-AcGIP, AcGIP, IV-AcGIP(LysPAL¹⁶), N-AcGIP(LysPAL³⁷), GIP(LysPAL¹⁶) or GIP(LysPAL³⁷). The peptide analogues can be used as a medicament in ameliorating or restoring age related decreased pancreatic beta-cell function, including glucose intolerance, loss of beta cell mass, beta-cell glucose insensitivity and/or insulin resistance.

The peptide analogues can also include the addition of linkers or residues to the N-terminal or C-terminal ends of the protein. A suitable linker includes, but is not limited to another active peptide or compound such as any of the therapeutically active molecules such as the anti-diabetogenic molecules described herein.

The peptide analogues can be used to screen compounds for their potential use as agonists or antagonists of the GIP receptor.

The peptide analogues can also be used to cause stem cells to differentiate into beta cells, to provide cell or replacement therapy for diabetes.

The invention also includes use of a peptide analogue of GIP in the manufacture of a medicament for ameliorating or restoring age related decreased pancreatic beta-cell function, including: glucose intolerance, beta-cell glucose insensitivity, and insulin resistance. The peptide analogue has at least 12 amino acid residues from the N-terminal end of GIP(1-42). The peptide analogue can include a further modification consisting of one of N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule. The peptide analogue can be N-Ac(GIP). The peptide analogue can alternatively or additionally be covalently attached to a polyethylene glycol (PEG) molecule. The peptide analogue can be in the form of a pharmaceutically acceptable salt, e.g., a pharmaceutically acceptable acid addition salt. The peptide analogue can also include modification by fatty acid addition at an epsilon amino group of at least one lysine residue, for instance, the modification can be the linking of a C-8 octanoyl group, C-10 decanoyl group, C-12 lauroyl group, C-14 myristoyl group, C-16 palmitoyl group, C-18 stearoyl group, or C-20 acyl group to the epsilon amino group of a lysine residue, for instance, the linking of a C-16 palmitoyl group to a lysine residue chosen from the group consisting of Lys¹⁶, Lys³⁰, Lys³², Lys³³ and Lys³⁷.

The peptide analogues, such as N-Ac(GIP), N-AcIP(LysPAL¹⁶), N-AcGIP(LysPAL³⁷), GIP(LysPAL¹⁶), GIP(LysPAL³⁷), can be used as a medicament. For instance, the peptide analogues can be used in the preparation of a medicament for ameliorating or restoring age related decreased pancreatic beta-cell function, such as the treatment of glucose intolerance, beta-cell glucose insensitivity, or insulin resistance. In such uses, the peptide analogue can be covalently attached to a polyethylene glycol (PEG) molecule.

Peptide analogues for use in the methods can include peptide analogues of GIP(1-42), comprising at least 12 amino acids from the N-terminal end of the protein.

The peptide analogues can also include analogues comprising at least 12 amino acid residues from the N-terminal end of GIP(1-42), and having an amino acid modification at any position, for example position 1 (such as N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule).

The peptide analogues used in the methods can include a modification by fatty acid addition at an epsilon amino group of at least one lysine residue, for instance, the modification can be the linking of a C-8 octanoyl group, C-10 decanoyl group, C-12 lauroyl group, C-14 myristoyl group, C-16 palmitoyl group, C-18 stearoyl group, or C-20 acyl group to the epsilon amino group of a lysine residue, for instance, the linking of a C-16 palmitoyl group to a lysine residue chosen from the group consisting of Lys¹⁶, Lys³⁰, Lys³², Lys³³ and Lys³⁷.

For instance, the peptide analogues useful in the methods can be a peptide analogue of GIP(1-42) (SEQ ID NO:1), wherein the analogue comprises: a base peptide consisting of one of the following: GIP(1-12), GIP(1-13), GIP(1-14), GIP(1-15), GIP(1-16), GIP(1-17), GIP(1-18), GIP(1-19), GIP(1-20), GIP(1-21), GIP(1-22), GIP(1-23), GIP(1-24), GIP(1-25), GIP(1-26), GIP(1-27), GIP(1-28), GIP(1-29), GIP(1-30), GIP(1-31), GIP(1-32), GIP(1-33), GIP(1-34), GIP(1-35), GIP(1-36), GIP(1-37), GIP(1-38), GIP(1-39), GIP(1-40), GIP(1-41), GIP(1-42); which base peptide possesses an amino acid substitution or modification at one or more of the residues. Suitable modifications include: (a) an amino acid substitution of lysine or cysteine for one or more or the residues, (b) an amino acid substitution or modification at position 1, (c) a modification by fatty acid addition at an epsilon amino group of at least one lysine residue, or (d) a modification by N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule, optionally, by N-terminal acetylation. The peptide analogue can be further modified by fatty acid addition at an epsilon amino group of at least one lysine residue, such as by the linking of a C-16 palmitoyl group to the epsilon amino group of a lysine residue, such as lysine residue Lys¹⁶ or lysine residue Lys³⁷.

For instance, the peptide analogues useful in the methods can be a peptide analogue of GIP(1-42) (SEQ ID NO:1), wherein the analogue comprises: a base peptide consisting of one of the following: GIP(1-12), GIP(1-13), GIP(1-14), GIP(1-15), GIP(1-16), GIP(1-17), GIP(1-18), GIP(1-19), GIP(1-20), GIP(1-21), GIP(1-22), GIP(1-23), GIP(1124), GIP(1-25), GIP(1-26), GIP(1-27), GIP(1-28), GIP(1-29), GIP(1-30), GIP(1-31), GIP(1-32), GIP(1-33), GIP(1-34), GIP(1-35), GIP(1-36), GIP(1-37), GIP(1-38), GIP(1-39), GIP(1140), GIP(1-41), GIP(142); which base peptide comprises:

(a) N-terminal glycation and an amino acid substitution or modification at one or both of positions 1 and 2
(b) amino acid substitution or modification at one or both of positions 1 and 2, where each amino acid substitution or modification is selected from the group consisting of:
(i) N-terminal alkylation;
(ii) N-terminal acetylation;
(iii) N-terminal acylation;
(iv) the addition of an N-terminal isopropyl group;
(v) the addition of an N-terminal pyroglutamic acid;
(vi) substitution at position 1 by a D-amino acid;
(vii) substitution at position 1 by an L-amino acid;
(viii) substitution at position 2 by a D-amino acid;
(ix) substitution at position 2 by L-alanine, L-arginine, L-asparagine, L-aspartic acid, L-cysteine, L-glutamic acid, L-glutamine, L-histidine, L-isoleucine, L-leucine, L-lysine, L-methionine, L-phenylalanine, L-proline, L-serine, L-threonine, L-tryptophan, L-tyrosine and L-valine;
(x) substitution at position 3 by a D-amino acid; and
(xi) substitution at position 3 by an L-amino acid; or
(c) amino acid substitution or modification at one of positions 1 and 2, where the amino acid substitution or modification is selected from the group consisting of:
(i) N-terminal alkylation;
(ii) N-terminal acetylation;
(iii) N-terminal acylation;
(iv) the addition of an N-terminal isopropyl group;
(v) the addition of an N-terminal pyroglutamic acid;
(vi) substitution at position 1 by a D-amino acid;
(vii) substitution at position 1 by an L-amino acid;
(viii) substitution at position 2 by D-arginine, D-asparagine, D-aspartic acid, D-cysteine, D-glutamic acid, D-glutamine, D-glycine, D-histidine, D-isoleucine, D-leucine, D-lysine, D-methionine, D-phenylalanine, D-proline, D-serine, D-threonine, D-tryptophan, D-tyrosine and D-valine;
(ix) substitution at position 2 by an L-amino acid;
(x) substitution at position 3 by a D-amino acid; and
(xi) substitution at position 3 by an L-amino acid.

For instance, the peptide analogues useful in the methods can be a peptide analogue of GIP(1-42) (SEQ ID NO:1), wherein the analogue comprises: a base peptide consisting of one of the following: GIP(1-12), GIP(1-13), GIP(1-14), GIP(1-15), GIP(1-16), GIP(1-17), GIP(1-18), GIP(1-19), GIP(1-20), GIP(1-21), GIP(1-22), GIP(1-23), GIP(1-24), GIP(1-25), GIP(1-26), GIP(1-27), GIP(1-28), GIP(1-29), GIP(1-30), GIP(1-31), GIP(1-32), GIP(1-33), GIP(1-34), GIP(11-35), GIP(1-36), GIP(1-37), GIP(1-38), GIP(1-39), GIP(140), GIP(1-41), GIP(1-42); A peptide analogue of GIP(1-42) which base peptide comprises:

(a) N-terminal glycation and an amino acid substitution or modification at one or more of positions 1 and 2;
(b) amino acid substitution or modification at one or both of positions 1 and 2, where each amino acid substitution or modification is selected from the group consisting of:
(i) N-terminal alkylation;
(ii) N-terminal acetylation;
(iii) N-terminal acylation;
(iv) the addition of an N-terminal isopropyl group;
(v) the addition of an N-terminal pyroglutamic acid;
(vi) substitution at position 1 by a D-amino acid;
(vii) substitution at position 1 by an L-amino acid;
(viii) substitution at position 2 by a D-amino acid;
(ix) substitution at position 2 by L-alanine, L-arginine, L-asparagine, L-aspartic acid, L-cysteine, L-glutamic acid, L-glutamine, L-histidine, L-isoleucine, L-leucine, L-lysine, L-methionine, L-phenylalanine, L-proline, L-serine, L-threonine, L-tryptophan, L-tyrosine and L-valine;
(x) substitution at position 3 by a D-amino acid; and
(xi) substitution at position 3 by an L-amino acid; or
(c) amino acid substitution or modification at one of positions 1 and 2, where the amino acid substitution or modification is selected from the group consisting of:
(i) N-terminal alkylation;
(ii) N-terminal acetylation;
(iii) N-terminal acylation;
(iv) the addition of an N-terminal isopropyl group;
(v) the addition of an N-terminal pyroglutamic acid;
(vi) substitution at position 1 by D-alanine, D-arginine, D-asparagine, D-aspartic acid, D-cysteine, D-glutamic acid, D-glutamine, D-glycine, D-histidine, D-isoleucine, D-leucine, D-lysine, D-methionine, D-phenylalanine, D-proline, D-serine, D-threonine, D-tryptophan and D-valine;
(vii) substitution at position 1 by an L-amino acid;
(viii) substitution at position 2 by D-arginine, D-asparagine, D-aspartic acid, D-cysteine, D-glutamic acid, D-glutamine, D-glycine, D-histidine, D-isoleucine, D-leucine, D-lysine, D-methionine, D-phenylalanine, D-proline, D-serine, D-threonine, D-tryptophan, D-tyrosine and D-valine;
(ix) substitution at position 2 by an L-amino acid;
(x) substitution at position 3 by D-alanine, D-arginine, D-asparagine, D-aspartic acid, D-cysteine, D-glutamic acid, D-glycine, D-histidine, D-isoleucine, D-leucine, D-lysine, D-methionine, D-phenylalanine, D-proline, D-serine, D-threonine, D-tryptophan, D-tyrosine and D-valine; and
(xi) substitution at position 3 by an L-amino acid.

Peptides useful in the present methods can be resistant to degradation by DPPIV relative to native GIP(1-42).

The invention also provides certain novel peptide analogues of GIP(1-42), wherein the peptide analogue is at least 12 amino acid residues from the N-terminal end of GIP(142) and wherein the peptide analogue comprises at least one amino acid substitution or modification, said at least one modification being fatty acid addition at an epsilon amino group of at least one lysine residue. When the said at least one amino acid substitution or modification is at a position other than positions 1, 2 or 3, DPPIV resistance is surprisingly noted. Alternatively, the novel peptide analogues can comprise a further at least one amino acid substitution or modification at one or both of positions 1 or 2 to increase receptor activation and, thereby, act as a GIP receptor agonist. The peptide analogue can further comprises an amino acid substitution of lysine for one or more of the residues and an amino acid modification by fatty acid addition at an epsilon amino group of said at least one substituted lysine residue. The peptide analogue can further comprise an amino acid substitution of cysteine for one or more of the residues and wherein the modification is the addition of a polyethylene glycol (PEG) molecule at said at least one substituted cysteine residue. The peptide analogue is a GIP agonist and wherein there can be an amino acid substitution or modification at one of more of positions 1, 2 and 3. The amino acid substitution, whether at one or more of positions 1, 2 or 3 (or indeed elsewhere) can be substituted by any L-amino acid selected from L-alanine, L-arginine, L-asparagine, L-aspartic acid, L-cysteine, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine. The amino acid substitution, whether at one or more of positions 1, 2 or 3 (or indeed elsewhere) can be substituted by any D-amino acid selected from by D-alanine, D-arginine, D-asparagine, D-aspartic acid, D-cysteine, D-glutamine, D-glycine, D-histidine, D-isoleucine, D-leucine, D-lysine, D-methionine, D-phenylalanine, D-proline, D-serine, D-threonine, D-tryptophan, D-tyrosine and D-valine. The or each, D-amino acid substitution can comprise replacement of the L-amino acid with its corresponding D-amino acid. Alternatively, the, or each, D-amino acid substitution can comprise replacement of the L-amino acid with any other D-amino acid. The amino acid substitution, whether at one or more of positions 1, 2 or 3 (or indeed elsewhere) can be substituted by any other L- or D-amino acid other than those commonly encountered in the genetic code, including beta amino acids such as beta-alanine and omega amino acids such as 3-amino propionic, 4-amino butyric, etc, ornithine, citrulline, homoarginine, t-butyl alanine, t-butyl glycine, N-methyl isoleucine, phenylglycine, cyclohexylalanine, norleucine, cysteic acid, and methionine sulfoxide. The peptide analogue can further comprise an amino acid modification at position 1, the N-terminal amino acid modification being optionally selected from N-terminal alkylation (using a saturated or unsaturated, straight or branched chain C¹⁻¹⁰ alkyl radical), N-terminal acylation (using, for example, a saturated or unsaturated, straight or branched chain C³⁻¹⁰ acyl radical), the addition of an N-terminal isopropyl group, the addition of an N-terminal pyroglutamic acid, or the addition of an N-terminal polyethylene glycol (PEG) molecule. The invention provides, in particular, peptide analogue being selected from GIP(LysPAL¹⁶), GIP(LysPAL³⁰), GIP(LysPAL³²), GIP(LysPAL³³) or GIP(LysPAL³⁷). The peptide analogue can be selected from GIP(LysPAL¹⁶) or GIP(LysPAL³⁷). The peptide analogue can further comprise an amino acid modification at position 1, the N-terminal amino acid modification being optionally selected from N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule.

The invention also provides certain novel peptide analogues of GIP(1-42), wherein the peptide analogue is at least 12 amino acid residues from the N-terminal end of GIP(1-42) and wherein the peptide analogue comprises at least one amino acid substitution or modification, wherein said at least one modification is the addition of a non-antigenic, water-soluble, biocompatible, inert polymer that prolongs the circulatory half-life of peptide analogue, for example, a polyethylene glycol (PEG) molecule. The peptide analogue can comprise either at least one modification being the addition of a polyethylene glycol (PEG) molecule at a position selected from the N-terminal position and the C-terminal position or at least one modification is the addition of a polyethylene glycol (PEG) molecule at a position other than a position selected from the N-terminal position and the C-terminal position. The peptide analogue can further comprises an amino acid substitution of cysteine for one or more of the residues and the modification can be the addition of a polyethylene glycol (PEG) molecule at said at least one substituted cysteine residue. The peptide analogue can further comprise an amino acid substitution of lysine for one or more of the residues and an amino acid modification by fatty acid addition at an epsilon amino group of said at least one substituted lysine residue. The peptide analogue can further comprise an amino acid modification at position 1, the N-terminal amino acid modification being optionally selected from N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule.

The base peptide for the novel peptide analogues of the invention can comprise GIP(1-12), GIP(1-13), GIP(1-14), GIP(1-15), GIP(1-16), GIP(1-17), GIP(1-18), GIP(1-19), GIP(1-20), GIP(1-21), GIP(1-22), GIP(1-23), GIP(1-24), GIP(1-25), GIP(1-26), GIP(1-27), GIP(1-28), GIP(1-29), GIP(1-30), GIP(1-31), GIP(1-32), GIP(1-33), GIP(1-34), GIP(1-35), GIP(1-36), GIP(1-37), GIP(1-38), GIP(1-39), GIP(1-40), GIP(1-41), GIP(1-42). This base peptide can possess at least one amino acid substitution or modification, wherein the at least one modification is fatty acid addition at an epsilon amino group of at least one lysine residue. The base peptide can alternatively possess an amino acid modification with a non-antigenic, water-soluble, biocompatible, inert polymer that prolongs the circulatory half-life of peptide analogue, for example, a PEG (optionally at its C-terminal end) and, optionally, a further amino acid modification at its N-terminal end. The N-terminal modification can be acylation (An acyl radical means a radical having a saturated or unsaturated, linear or branched aliphatic chain, of from 4 to 22 carbons), optionally, acetylation.

The peptide analogue can selected from N-AcGIP(1-12)(PEG), N-AcGIP(1-13)(PEG), N-AcGIP(1-14)(PEG), N-AcGIP(1-15)(PEG), N-AcGIP(1-16)(PEG), N-AcGIP(1-17)(PEG), N-AcGIP(1-18)(PEG), N-AcGIP(1-19)(PEG), N-AcGIP(1-20)(PEG), N-AcGIP(1-21)(PEG), N-AcGIP(1-22)(PEG), N-AcGIP(1-23)(PEG), N-AcGIP(1-24)(PEG), N-AcGIP(1-25)(PEG), N-AcGIP(1-26)(PEG), N-AcGIP(1-27)(PEG), N-AcGIP(1-28)(PEG), N-AcGIP(1-29)(PEG), N-AcGIP(1-30)(PEG), N-AcGIP(1-31)(PEG), N-AcGIP(1-32)(PEG), N-AcGIP(1-33)(PEG), N-AcGIP(1-34)(PEG), N-AcGIP(1-35)(PEG), N-AcGIP(1-36)(PEG), N-AcGIP(1-37)(PEG), N-AcGIP(1-38)(PEG), N-AcGIP(1-39)(PEG), N-AcGIP(1-40)(PEG), N-AcGIP(1-41)(PEG), N-AcGIP(1-42)(PEG), wherein the peptide analogue is optionally N-AcGIP(PEG).

The invention also provides a pharmaceutical composition comprising a novel peptide analogue, in association with a pharmaceutically acceptable carrier. The pharmaceutical composition may further comprise a therapeutically effective amount of an agent having an antidiabetic effect.

Any of the peptide analogues described herein can also be non-human derived versions of the GIP protein. For instance, the peptide analogues can be analogues of the GIP protein as it is found in rat, mouse, hamster, sheep, cow, pig, goat, dog, cat, etc.

Any of the peptide analogues described herein can be included in a pharmaceutical composition. Such a pharmaceutical composition can include a pharmaceutically acceptable carrier. The peptide analogues can be in the form of a pharmaceutically acceptable salt, and/or a pharmaceutically acceptable acid addition salt.

The peptide analogues can be combined with other treatment regimens, for instance, the peptide analogues can be combined with antidiabetic treatments, such as biguanides (such as, but not limited to, metformin), sulphonylureas (such as, but not limited to, acetohexamide, chlorpropamide, tolbutamide, tolazamide, glimepiride, gliclazide, glipizide, glyburide, glibenclamide), thiazolidinediones (also called glitazones) (such as, but not limited to, pioglitazone (e.g., pioglitazone hydrochloride), rosiglitazone (rosiglitazone maleate), troglitazone), meglitinides (such as, but not limited to, nateglinide, repaglinide), alpha-glucosidase inhibitors (such as, but not limited to, acarbose, miglitol), incretin mimetics (such as, but not limited to, exenatide), amylinomimetics (such as, but not limited to, prarnlintide acetate). The peptide analogues can also be combined with other peptide molecules, such as GLP-1, or analogues of such peptide molecules.

The peptide analogues used in the invention are preferably resistant to degradation by enzyme DPP IV, when compared to naturally-occurring GIP.

The methods provided herein are useful in treating symptoms of decreased pancreatic beta-cell function, including decreased pancreatic beta-cell function, including glucose intolerance, loss of beta-cell mass (e.g. treatment by inducing neogenesis or proliferation of beta cells or inhibition of apoptosis of beta cells), beta-cell glucose insensitivity, insulin resistance and reduced insulin secretion.

### BRIEF DESCRIPTION OF THE DRAWINGS

FIGS. 1A-1D are a set of four bar graphs showing the effects of age on food intake (FIG. 1A), body weight (FIG. 1B), plasma glucose (FIG. 1C) and insulin (FIG. 1D) concentrations in young (white bars) versus old (black bars) mice. Values are mean±SEM for six mice. *P<0.05, **P<0.01, ***P<0.001 compared with younger mice.

FIGS. 2A-2D are line graphs (FIGS. 2A, 2B) and bar graphs (FIGS. 2C, 2D) showing the effects of age on glucose tolerance (FIGS. 2A, 2C) and plasma insulin response to glucose (FIGS. 2B, 2D) in young (□; white bars) and old (▴; black bars) mice. Glucose (18 mmol/kg body weight) was administered by intraperitoneal injection at the time indicated by the arrow. Plasma glucose and insulin AUC values for 0-60 minutes post injection are also shown. Values are mean±SEM for six mice. *P<0.05, **P<0.01 compared with younger mice.

FIGS. 3A-3D are line graphs (FIGS. 3A, 3B) and bar graphs (FIGS. 3C, 3D) showing the effects of age on glucose tolerance (FIGS. 3A, 3C) and plasma insulin response to glucose (FIGS. 3B, 3D) in young (□; white bars) and old (▴; black bars) mice. Glucose (18 mmol/kg body weight) in combination with native GIP (25 nmol/kg bodyweight) was administered by intraperitoneal injection at the time indicated by the arrow. Plasma glucose and insulin AUC values for 0-60 minutes post injection are also shown. Values are mean±SEM for six mice. *P<0.05 compared with younger mice.

FIGS. 4A-4D are line graphs (FIGS. 4A, 4B) and bar graphs (FIGS. 4C, 4D) showing the effects of N-AcGIP(LysPAL³⁷) and age on glucose tolerance (FIGS. 4A, 4C) and plasma insulin response to glucose (FIGS. 4B, 4D) in young (□; white bars) and old (▴; black bars) mice. Glucose (18 mmol/kg body weight) in combination with N-AcGIP(LysPAL³⁷) (25 nmol/kg bodyweight) was administered by intraperitoneal injection at the time indicated by the arrow. Plasma glucose and insulin AUC values for 0-60 min post injection are also shown. Values are mean±SEM for six mice. *P<0.05, **P<0.01, ***P<0.001 compared with younger mice.

FIGS. 5A and 5B are a pair of bar graphs showing ratio of plasma glucose (FIG. 5A) and insulin (FIG. 5B) AUC values for native GIP and N-AcGIP(LysPAL³⁷) compared to glucose alone in younger (white bars) and older (black bars) adult mice. Plasma glucose and insulin concentrations were measured prior to and after i.p. administration of glucose alone (18 mmol/kg) or in combination with GIP or N-AcGIP(LysPAL³⁷) (25 nmol/kg). Ratios are calculated from AUC data illustrated in FIGS. 2-4. Data represent means±SEM for six mice. **P<0.05 compared to young mice.

FIG. 6 is a bar graph showing the effects of no GIP (left two bars) or 10 nM GIP treatment (right two bars) on insulin secretion from BRIN-BD11 cells exposed to 5.6 mM glucose (white bars) or 5.6 mM glucose and 200 μM tolbutamide (black bars). Values are percentage±SEM (relative to control group (5.6 mmol/l glucose alone) for eight separate observations. ***P<0.001 compared to respective effect without GIP. ^{ΔΔΔ}P<0.001 compared to corresponding test in absence of tolbutamide.

FIG. 7 is a bar graph showing the effects of no GIP (left two bars) or 10 nM GIP treatment (right two bars) on insulin secretion from BRIN-BD11 cells exposed to 5.6 mM glucose (white bars) or 5.6 mM glucose and 200 μM glibenclamide (black bars). Values are percentage±SEM (relative to control group (5:6-mmol/l glucose alone) for eight separate observations. ***P<0.001 compared to respective effect without GIP. ^{ΔΔ}P<0.01, ^{ΔΔΔ}P<0.001 compared to corresponding test in absence of glibenclamide.

FIG. 8 is a bar graph showing the effects of no GIP (left two bars) or 10 nM GIP treatment (right two bars) on insulin secretion from BRIN-BD11 cells exposed to 5.6 mM glucose (white bars) or 5.6 mM glucose and 200 μM nateglinide (black bars). Values are percentage±SEM (relative to control group (5.6 mmol/l glucose alone) for eight separate observations. ***P<0.001 compared to respective effect without GIP. ^{ΔΔΔ}P<0.001 compared to corresponding test in absence of nateglinide.

FIG. 9 is a bar graph showing the effects of no GIP (left two bars) or 10 nM GIP treatment (right two bars) on insulin secretion from BRIN-BD11 cells exposed to 5.6 mM glucose (white bars) or 5.6 mM glucose and 25 μM troglitazone (black bars). Values are percentage±SEM (relative to control group (5.6 mmol/l glucose alone) for eight separate observations. ***P<0.001 compared to respective effect without GIP. ^{ΔΔΔ}P<0.001 compared to corresponding test in absence of troglitazone.

FIG. 10 is a bar graph showing the effects of no GIP (left three bars) or 10 nM GIP treatment (right three bars) on insulin secretion from BRIN-BD11 cells exposed to 5.6 mM glucose (white bars), 5.6 mM glucose and 50 μM metformin (grey bars), or 5.6 mM glucose and 200 μM metformin (black bars). Values are percentage±SEM (relative to control group (5.6 mmol/glucose alone) for eight separate observations. ***P<0.001 compared to respective effect without GIP. ^{ΔΔΔ}P<0.001 compared to corresponding test in absence of metformin.

FIG. 11 is a line graph showing intracellular cAMP production by GIP(□), GIP(LysPAL¹⁶) () and GIP(LysPAL³⁷) (▾) as determined by column chromatography, in CHL cells stably expressing the human GIP receptor. Each experiment was performed in triplicate (n=3) and the results expressed (means±SEM) as a percentage of maximum GIP response.

FIG. 12 is a bar graph showing the insulin-releasing activity of GIP (black bars), GIP(LysPAL¹⁶) (diagonally cross-hatched bars) and GIP(LysPAL³⁷) (gridded bars) in the clonal pancreatic beta cell line, BRIN-BD11, relative to control treatment with 5.6 mM glucose (white bars). After a pre-incubation (40 min), the effects of various concentrations of peptide were tested on insulin release during a 20 minute incubation. Values are means±SEM for 8 separate observations. *P<0.05, **P<0.01, ***P<0.001 compared to control (5.6 mM glucose alone).

FIGS. 13A and 13B are a line graph and a bar graph, respectively, showing the glucose lowering effects of GIP (□, black bars), GIP(LysPAL^{≠}) (, diagonally cross-hatched bars) and GIP(LysPAL³⁷) (▾, gridded bars) in 18 hour fasted (ob/ob) mice, relative to treatments with glucose alone (⋄, white bars). Plasma glucose concentrations (FIG. 13A) were measured prior to and after intraperitoneal administration of glucose alone (18 mM kg⁻¹ body weight) as a control, or in combination with GIP or fatty acid derivatised analogues (25 nmol kg⁻¹ body weight). The incremental area under the glucose curve (AUC) between 0 and 60 minutes is shown in FIG. 13B. Values represent means±SEM for 8 mice. *P<0.05, **P<0.01 compared to glucose alone. ^{Δ}P<0.05 compared to native GIP.

FIGS. 14A and 14B are a line graph and a bar graph, respectively, showing the insulin releasing activity of GIP (□, black bars), GIP(LysPAL¹⁶) (, diagonally cross-hatched bars) and GIP(LysPAL³⁷) (▾, gridded bars) in 18 hour fasted (ob/ob) mice, relative to treatments with glucose alone (⋄, white bars). FIG. 14A shows plasma insulin concentrations, which were measured prior to and after intraperitoneal administration of glucose alone (18 mM kg⁻¹ body weight) as a control, or in combination with GIP or fatty acid derivatised analogues (25 nmol kg⁻¹ body weight). The incremental area under the insulin curve (AUC) between 0 and 60 minutes is shown in FIG. 14B. Values represent means±SEM for 8 mice. *P<0.05, **P<0.01, ***P<0.001 compared to glucose alone. ^{Δ}P<0.05, ^{ΔΔ}P<0.01 compared to native GIP.

FIGS. 15A and 15B are a line graph and a bar graph, respectively, showing the prolonged glucose lowering effects of GIP (□, black bars) and GIP(LysPAL³⁷) (▾, gridded bars) in ob/ob mice, relative to saline-treated controls (⋄, white bars). FIG. 15A shows the twenty four hour plasma glucose concentration profile after intraperitoneal administration of saline alone (0.9% w/v NaCl) as a control, GIP or GIP(LysPAL³⁷) (12.5 nmol kg⁻¹ body weight). FIG. 15B shows the incremental area under the glucose curve (AUC) between 0 and 24 hours. Values represent means±SEM for 8 mice. *P<0.05 and **P<0.01 compared to saline alone. ^{Δ}P<0.05 and ^{ΔΔ}P<0.01 compared to native GIP.

FIGS. 16A and 16B are a line graph and a bar graph, respectively, showing the prolonged insulinotropic effects of GIP (□, black bars) and GIP(LysPAL³⁷) (▾, gridded bars) in ob/ob mice, relative to saline-treated controls (⋄, white bars). FIG. 16A shows the twenty four hour plasma insulin concentration profile after intraperitoneal administration of saline alone (0.9% w/v NaCl) as a control, GIP or GIP(LysPAL³⁷) (12.5 nmol kg⁻¹ body weight). FIG. 16B shows the incremental area under the insulin curve (AUC) between 0 and 24 hours. Values represent means±SEM for 8 mice.

FIGS. 17A-17D are two line graphs and two bar graphs showing the effects of N-AcGIP, N-AcGIP(LysPAL³⁷) and GIP(LysPAL³⁷) on plasma glucose (FIGS. 17A, 17B) and insulin response (FIGS. 17C, 17D) 4 hours after administration. Tests were conducted 4 hours after administration of N-AcGIP, N-AcGIP(LysPAL³⁷), GIP(LysPAL³⁷) (25 mmoles/kg) or saline (0.9% NaCl) in 18 hour-fasted ob/ob mice. Plasma glucose and insulin concentrations (FIGS. 17A, 17C, respectively) were measured prior to and after i.p. administration of glucose alone (18 mmoles/kg). The incremental area under the glucose or insulin curves (AUC) between 0 and 60 min are shown in the lower panels (FIGS. 17B, 17D, respectively). Values represent means±SEM for 8 mice. *p<0.05 and **p<0.01 compared with saline alone group.

FIGS. 18A-18E show the effects of daily N-AcGIP, N-AcGIP(LysPAL³⁷) and GIP(LysPAL³⁷) administration on body weight (FIG. 18A), food intake (FIG. 18B), plasma glucose (FIG. 18C), plasma insulin (FIG. 18D) and final glucagon levels (FIG. 18E). N-AcGIP (A, diagonally cross-hatched bar), N-AcGIP(LysPAL³⁷) (V, black bar), GIP(LysPAL³⁷) (♦, horizontally cross-hatched bar) (25 nmole's/kg/day) or saline vehicle (control, □, white bar) were administered for the 14-day period indicated by the horizontal black bar. Values are mean±SEM for 8 mice. *p<0.05 compared to control.

FIGS. 19A-19D are a set of two line graphs (FIGS. 19A, 19C) and bar graphs (FIGS. 19B, 19D) showing the effects of daily saline (□; white bars) N-AcGIP (Δ, diagonally cross-hatched bars), N-AcGIP(LysPAL³⁷) (▾, black bars) and GIP(LysPAL³⁷) (horizontally cross-hatched bars) administration on glucose tolerance (FIGS. 19A, 19B) and plasma insulin response to glucose (FIGS. 19C, 19D). Tests were conducted after 14 daily injections of either N-AcGIP, N-AcGIP(LysPAL³⁷), GIP(LysPAL³⁷) (25 nmoles/kg/day) or saline vehicle (control). Glucose (18 mmoles/kg) was administered by intraperitoneal injection at the time zero. Plasma glucose and insulin AUC values for 0-60 min post injection are shown in the lower panels. Values are mean±SEM for 8 mice. *p<0.05 and **p<0.01 compared to control.

FIGS. 20A-20D are a set of two line graphs (FIGS. 20A, 20C) and bar graphs (FIGS. 20B, 20D) showing the effects of daily N-AcGIP (Δ), N-AcGIP(LysPAL³⁷) (▾, black bars) and GIP(LysPAL³⁷) (♦) administration on insulin sensitivity. Tests were conducted after 14 daily injections of either N-AcGIP, N-AcGIP(LysPAL³⁷), GIP(LysPAL³⁷) (25 nmoles/kg/day) or saline vehicle (control). Insulin (50 U/kg) was administered by intraperitoneal injection at the time zero. Plasma glucose as percent basal and mmol/l are shown in FIGS. 20A and 20C. Plasma glucose AUC values for 0-60 min post injection are shown in the lower panels (FIGS. 20B, 20D). Values are mean±SEM for 8 mice. *p<0.05 and **p<0.01 compared to control.

FIGS. 21A-21D are a set of two line graphs (FIGS. 21A, 21C) and bar graphs (FIGS. 21B, 21D) showing the effects of daily N-AcGIP (A, diagonally cross-hatched bars), N-AcGIP(LysPAL³⁷) (▾, black bars) and GIP(LysPAL³⁷) (⋄, horizontally cross-hatched bars) administration on glucose (FIGS. 21A, 21B) and insulin (FIGS. 21C, 21D) responses to feeding in 18 hours fasted ob/ob mice. Tests were conducted after daily treatment with N-AcGIP, N-AcGIP(LysPAL³⁷), GIP(LysPAL³⁷) (25 nmoles/kg/day) or saline for 14 days. The time of feeding was at 15 minutes. Plasma glucose and insulin concentrations are shown in FIGS. 21A and 21C, respectively. AUC values for 0-105 min post-feeding are shown in FIGS. 21B and 21D. Values are mean±SEM for eight mice. *p<0.05 and **p<0.01 compared to control.

FIGS. 22A-D are a set of two line graphs (FIGS. 22A, 22C) and bar graphs (FIGS. 22B, 22D) showing the effects of daily N-AcGIP (Δ, diagonally cross-hatched bars), N-AcGIP(LysPAL³⁷) (▾, black bars) and GIP(LysPAL³⁷) (♦, horizontally cross-hatched bars) administration on glucose tolerance (FIGS. 22A, 22B) and plasma insulin response (FIGS. 22C, 22D) to native GIP. Tests were conducted after 14 daily injections of either N-AcGIP, N-AcGIP(LysPAL³⁷), GIP(LysPAL³⁷) (25 nmoles/kg/day) or saline vehicle (control, □, white bars). Glucose (18 mmoles/kg) in combination with native GIP (25 mmoles/kg) was administered by intraperitoneal injection at the time indicated by the arrow. Plasma glucose and insulin AUC values for 0-60 min post injection are shown in the right panels. Values are mean±SEM for 8 mice. *p<0.05, **p<0.01 and ***p<0.001 compared to control.

FIGS. 23A and 23B are a pair of bar graphs showing the effects of daily N-AcGIP (diagonally cross-hatched bars), N-AcGIP(LysPAL³⁷) (black bars) and GIP(LysPAL³⁷) (horizontally cross-hatched bars) administration on pancreatic weight (FIG. 23A) and insulin content (FIG. 23B), relative to saline treated controls (white bars). Parameters were determined after 14 daily injections of N-AcGIP, N-AcGIP(LysPAL³⁷), GIP(LysPAL³⁷) (25 nmoles/kg/day) or saline vehicle (control). Values are mean±SEM for 8 mice. *p<0.05 compared to control.

FIGS. 24A and 24B are a pair of bar graphs showing the effects of daily N-AcGIP (diagonally cross-hatched bars), N-AcGIP(LysPAL³⁷) (black bars) and GIP(LysPAL³⁷) (horizontally cross-hatched bars) administration on islet area (FIG. 24A) and islet number (FIG. 24B), relative to saline treated controls (white bars). Parameters were determined after 14 daily injections of N-AcGIP, N-AcGIP(LysPAL³⁷), GIP(LysPAL³⁷) (25 nmoles/kg/day) or saline vehicle (control). Values are mean±SEM for 8 mice. *p<0.05, **p<0.01 compared to control.

FIGS. 25A and 25B are a pair of bar charts showing the effects of various glucose concentrations on insulin secretion from differentiated D3 cluster cells under various conditions. FIG. 25A shows these effects in Stage 4 cells and FIG. 25B shows these effects in Stage 5 cells. Acute incubations were performed at 5.6 mM glucose (control) or varying levels of glucose (0 mM, 16.7 mM, 22.0 mM). Left-hand group shows glucose control treatments, media of right-hand group was supplemented with 1×10⁻⁶ M GIP(LysPAL¹⁶). Values are the mean±SEM for 8 separate observations. *P<0.05, **P<0.01 and **P<0.001 compared with 5.6 mM glucose alone under same culture conditions. ^{Δ}P<0.05, ^{ΔΔ}P<0.01 and ^{ΔΔΔ}P<0.001 compared with respective test reagent following control culture.

FIGS. 26A and 26B are a pair of bar graphs showing the effects of various secretagogues on insulin secretion from differentiated D3 cluster cells under different conditions, with FIG. 26A showing these effects in Stage 4 cells and FIG. 26B showing Stage 5 cells. Acute incubations were performed at 5.6 mM glucose. Other treatments included 10 mM alanine, 25 μM forskolin, 10 nM PMA, and 7.4 mM CaCl₂. Left-hand group shows control treatments, media of right-hand group was supplemented with 1×10-6 M GIP(LysPAL¹⁶). Values are the mean±SEM for 8 separate observations. **P<0.01 and ***P<0.001 compared with 5.6 mM glucose alone under same culture conditions. ^{ΔΔ}P<0.01 and ^{ΔΔΔ}P<0.001 compared with respective test reagent following control culture.

FIG. 27 is a pair of line graphs showing the effects of daily N-AcGIP(PEG) administration on food intake and body weight. N-AcGIP(PEG) (25 nmoles/kg/day) or saline vehicle (control) were administered for the 14-day period. Values are mean±SEM for 8 mice.

FIG. 28 is a pair of line graphs showing changes of plasma glucose and insulin after daily treatment of ob/ob mice with N-AcGIP(PEG) (25 nmoles/kg/day) or saline vehicle (control) for 14 days. Values are mean±SEM for 8 mice.

FIG. 29 is a set of line graphs showing the effects of daily N-AcGIP(PEG) administration on glucose tolerance and plasma insulin response to glucose. Tests were conducted after 14 daily injections of either N-AcGIP(PEG) (25 mmoles/kg/day) or saline vehicle (control). Glucose (18 mmoles/kg) was administered by intraperitoneal injection. Plasma glucose and insulin AUC values for 0-60 min post injection are shown in the right panels. Values are mean±SEM for 8 mice. *p<0.05 compared to control.

FIG. 30 is a line graph and a bar graph showing the effects of daily N-AcGIP(PEG) administration on insulin sensitivity. Tests were conducted after 14 daily injections of either N-AcGIP(PEG) (25 nmoles/kg/day) or saline vehicle (control). Insulin (50 U/kg) was administered by intraperitoneal injection. Plasma glucose AUC values from baseline for 0-60 min post injection are shown in the right panels. Values are mean±SEM for 8 mice. *p<0.05 compared to control.

### DETAILED DESCRIPTION

Methods and peptide analogues are provided for treating age-related symptoms of decreased pancreatic beta-cell function, including glucose intolerance, beta-cell glucose insensitivity, insulin resistance and reduced insulin secretion. The peptide analogues are analogues of gastric inhibitory peptide (GIP), such as N-AcGIP, GIP(LysPAL¹⁶), GIP(LysPAL 37) and N-AcGIP(LysPAL³⁷). GIP(LysPAL¹⁶), GIP(LysPAL³⁷) and N-AcGIP(LysPAL³⁷) are analogues of gastric inhibitory peptide (GIP), which are either acetylated or not acetylated at the N-terminus, and are modified by a fatty acid (a C-16 palmitoyl radical) addition at an epsilon amino group of the lysine residue at position 16 or 37 of native GIP. The peptide analogues act as agonists of native GIP.

As used herein, an “agonist” is a peptide analogue of GIP, which activates the GIP receptor, or otherwise mimics, prolongs or enhances the biological activity shown by native GIP, such as by potentiating glucose-induced insulin secretion. Glucose tolerance tends to progressively decline with age. Glucose-dependent insulinotropic polypeptide (GIP) potentiates glucose-induced insulin secretion. As shown herein, the GIP receptor agonist N-AcGIP(LysPAL³⁷) shows potent, long-lasting insulinotropic effects in aging mice.

In older mice, body weights, basal plasma glucose and insulin concentrations were significantly higher than in young controls (P<0.05 to P<0.001). Intraperitoneal injection of glucose alone (18 nmol/kg body weight) revealed a significantly lower (P<0.05) insulin response in older mice, which was accompanied by impaired glucose tolerance (P<0.05). Normal glucose-mediated insulin secretion was restored in N-AcGIP(LysPAL³⁷) treated older mice when compared to younger adults. However the glycaemic excursion remained significantly impaired in older mice (P<0.05), suggestive of impaired insulin action. Native GIP had a similar overall effect in younger and older mice. These data indicate that peptide analogues such as N-AcGIP(LysPAL³⁷) are able to counter the age-related deterioration of pancreatic beta cell glucose sensitivity and insulin secretion, and that stable GIP agonists are valuable in therapy and combination therapy of age-related deterioration of pancreatic beta cell glucose sensitivity. The two most important insulin-releasing hormones secreted from endocrine cells are glucose-dependent insulinotropic polypeptide (gastric inhibitory polypeptide; GIP) and glucagon-like peptide-1 (GLP-1). (GIP) is a 42 amino acid peptide released form the intestinal K-cells in response to oral nutrient ingestion (Buchan, M. T. et al., 1978, Histochemhistry 56:37-44), and is believed to play a key role in the early phase insulin secretory response to glucose and meal ingestion (Lewis, J. T. et al., 2000, Endocrinology 141:3710-3716). Following postprandial release into the circulation, the primary role of GIP is to help maintain normal glucose homeostasis through glucose-dependent insulin release (Creutzfeldt, W., 2001, Exp. Clin. Endocrinol. Diabetes 109:S288-303). Beta-cell function progressively declines with age (Resnick, H. E. et al., 2000, Diabetes Care 23:176-180), and beta-cell sensitivity to GIP is thought to be impaired in the elderly (Elahi, D. et al., 1984, Diabetes 33:950-957).

GIP stimulates proinsulin gene transcription and translation (Fehmann, H. C. et al., 1995, Endocrinol. Rev. 16:390-410; Wang, Y. et al., 1996, Mol. Cell. Endocrinol. 116:81-87) and also acts synergistically as both a growth and anti-apoptotic factor for pancreatic beta cells (Trumper, A. et al., 2001, Mol. Endocrinol. 15:1559-1570; Trumper, A. et al., 2002, J. Endocrinol. 174:233-246; Ehses, J. A. et al., 2003, Endocrinol. 144:4433-4445).

Native GIP is rapidly degraded in the circulation by the ubiquitous enzyme dipeptidyl peptidase IV (DPP IV; EC 3.4.15.5), yielding a truncated GIP metabolite, GIP(3-42) (Gault, V. A. et al., 2002, J. Endocrinol. 175:525-533). DPP IV activity was shown to be similar in healthy elderly and middle-aged patients (Meneilly, G. S. et al., 2000, Diabet. Med. 17:6-30). Native GIP is also rapidly cleared from the body by the kidney tubules (Meier, J. J. et al., 2004, Diabetes 53:654-662). N-AcGIP(LysPAL³⁷) is a fatty acid derivatised, N-terminally modified GIP analogue which has been shown to exhibit prolonged bioactivity due to resistance to both enzymatic degradation and renal clearance (Irwin, N. et al., 2005, Biol. Chem. (In Press)). N-AcGIP(LysPAL³⁷) is an example of a long-acting analogue of GIP with beneficial actions in correcting the impairment of insulin secretion and glucose tolerance associated with advanced age.

Recent data have shown that GLP-1 is capable of countering the age-related decline in beta-cell function, but its use is hindered by its short biological half life (Doyle, M. E., and Egan, J. M., 2001, Recent Prog. Horm. Res. 56:377-399). As shown herein, N-AcGIP(LysPAL³⁷) significantly augmented the overall glucose-mediated insulin response in older mice as compared to younger controls, indicating that GIP stimulation can help overcome the beta-cell defect associated with aging (Elahi, D. et al., 1984, Diabetes 33:950-957). However, the overall glycaemic excursion in the N-AcGIP(LysPAL³⁷) treated older mice was significantly elevated, in a similar fashion to that observed following injection with glucose alone. This signifies that the stable GIP analogue did not overcome the accompanying decrease of insulin sensitivity present in older mice (Bailey, C. J., and Flatt, P. R., 1982, Int. J. Obesity 6:11-21). Although little is known about the regulation of receptors for GIP on islet cells, the cAMP-mediated insulin secretion pathway through which GIP and other insulin secretagogues operate has been shown to be impaired with aging (Aizawa, T. et al., 1994, Pancreas 9:454-459), pointing towards a more generalized rather than a GIP-specific beta-cell defect lesion (Elahi, D. et al., 1984, Diabetes 33:950-957). Some desensitization was however apparent as native GIP had no beneficial insulinotropic effects in older mice when compared to younger controls.

The actions of N-AcGIP(LysPAL³⁷) were evident in the present study despite the presence of significantly elevated non-fasting circulating plasma insulin concentrations in older adult mice. Non-fasting plasma glucose levels were also shown to be significantly elevated in older adult mice evidencing the insulin resistance generally present in the elderly (Elahi, D. et al., 2002, Novartis Found. Symp. 242:222-242). At the ages studied, food intake was similar in the two groups of mice, but one difference between younger and older mice was the increase in body weight. This is likely to contribute to the age-related metabolic decline, as increased adiposity has long been associated with insulin resistance and compensatory hyperinsulinaemia (Raven, G. M., 1991, Am. Heart J. 121:1283-1288).

Several GIP analogues with improved pharmacokinetic properties have been generated, and the characteristics and antidiabetic actions of these GIP analogues, N-AcGIP, GIP(LysPAL¹⁶), GIP(LysPAL³⁷) and N-AcGIP(LysPAL³⁷), are disclosed herein.

The extended duration of action and DPP IV resistance of the GIP analogues were confirmed prior to evaluation of once daily injections over a 14-day period. Administration of either N-AcGIP, GIP(LysPAL³⁷) or N-AcGIP(LysPAL³⁷) significantly decreased non-fasting plasma glucose and improved glucose tolerance. N-AcGIP, GIP(LysPAL³⁷) or N-AcGIP(LysPAL³⁷) treatment caused a significant enhancement in the insulin response to intraperitoneal glucose or nutrient intake, together with a notable improvement of insulin sensitivity. The metabolic responses to native GIP were enhanced in all the 14-day GIP analogue treated mice revealing no evidence of GIP-receptor desensitization or down regulation. In addition, beta cell mass was increased by all three analogues and ability of N-AcGIP(LysPAL¹⁶) to direct embryonic stem cell differentiation towards beta cell phenotype was demonstrated. The data demonstrate the utility of N-terminally modified GIP analogues, acylated derivatives of GIP and general strategies to promote binding of GIP to circulating proteins as potential agents for age-related deterioration of pancreatic beta cell glucose sensitivity.

Recent research has elucidated that the two gut hormones, glucagon-like peptide-1 (GLP-1) and glucose dependent insulinotropic polypeptide (GIP), act as incretin hormones and account for predominant part of the postprandial insulin response. In addition to enhancing insulin secretion, GIP has been shown to up-regulate proinsulin gene transcription and translation (Wang, Y. et al., 1996, Mol. Cell. Endocrinol. 116:81-87), increase pancreatic β cell growth (Kim, S. J. et al., 2005, J Biol. Chem. 280: 22297-22307), and inhibit pancreatic β cell apoptosis (Ehses, J. A. et al., 2003, Endocrinology 144: 4433-4445).

Owing to its potent glucose-dependent insulinotropic actions, GIP has been suggested as a possible therapeutic option for the treatment of age-related deterioration of pancreatic beta cell glucose sensitivity. The peptidic nature and unfavorable pharmacokinetic profile have somewhat hindered the therapeutic progression of GIP. As a consequence, work on bioengineered GIP analogues exhibiting different pharmacokinetic properties and biological potency has begun (Green, B. D. et al., 2004, Curr. Pharm. Des. 10: 3651-3662). With the advent of new designer, bioengineered longer-acting GIP molecules (Gault, V. A. et al., 2002, Biochem. J. 367: 913-920; O'Harte, F. P. M. et al., 2002, Diabetologia 45:1281-1291; Irwin, N. et al. 2005, J. Med. Chem. 48:1244-1250; Irwin, N. et al., 2005, J. Pharm. Expt. Therap. (in press)) a convincing rationale for future diabetes treatment strategies based on GIP now exists.

Although the glucose-dependent insulinotropic action of GIP was discovered almost 30 years ago (Pederson, R. A. and Brown, J. C., 1976, Endocrinology 99:780-785), therapeutic strategies based on GIP are not yet available. The polypeptide structure of GIP necessitates parenteral administration. Furthermore, GIP is rapidly degraded by the ubiquitous enzyme dipeptidylpeptidase IV (DPP IV) yielding the major degradation fragment GIP(342), which lacks insulinotropic activity (Gault, V. A. et al., 2002, J. Endocrinol. 175: 525-533). Therefore, the development of DPP IV resistant analogues of GIP would not only extend the half-life and increase biological potency of the peptide. However, it is important to note that the in vivo half-life of GIP not only depends on enzymatic degradation but also renal elimination. One method to delay renal extraction of GIP is through covalent linkage of a free fatty acid chain, to promote plasma albumin binding Tyr¹ modified analogues of GIP have been developed, modeled on previous studies with the glucagon-secretin family of gastrointestinal peptides, which exhibit profound resistance to DPP IV (Green, B. D. et al., 2004, Curr. Pharm. Des. 10: 3651-3662). As a result of degradation resistance and enhanced in vitro activity, these analogues displayed notable antidiabetic promise when administered acutely to obese diabetic (ob/ob) mice (Green, B. D. et al., 2004, Curr. Pharm. Des. 10: 3651-3662). Furthermore, GIP analogues utilizing secondary modifications such as fatty acid derivatization, which counters renal clearance by promoting albumin binding of peptide, have been generated (Irwin, N. et al. 2005, J. Med. Chem. 48:1244-1250; Irwin, N. et al., 2005, J. Pharm. Expt. Therap. (in press)). As a result of fatty acid derivatisation, these analogues displayed-pharmacodynamic properties suitable for once daily administration for the treatment of type 2 diabetes.

The present study was designed to examine the glucose tolerance and insulin resistance effects of extended treatment with three longer-acting GIP analogues namely, N-AcGIP, GIP(LysPAL³⁷) and N-AcGIP(LysPAL³⁷). Experiments were also conducted to determine the DPP IV resistance and both cellular and acute metabolic effects of the novel GIP(LysPAL¹⁶) and GIP(LysPAL³⁷) analogues. The ability of structural GIP agonists to increase pancreatic beta cell mass and promote differentiation of stem cells towards beta cell phenotype were also evaluated. Overall, the results indicate enhanced potential of both N-terminal modification and mid-chain acylation of native GIP.

As shown herein, successful synthesis of fatty acid derivatised GIP analogues with ability to bind serum albumin were confirmed by MALDI-TOF mass spectrometry. Surprisingly, both GIP(LysPAL¹⁶) and GIP(LysPAL³⁷) displayed resistance to DPP IV, which rapidly cleaved native GIP to the inactive metabolite GIP(3-42). Thus, whereas in vitro degradation of native GIP was substantial by 2 hours and complete within 8 hours, both fatty acid derivatised analogues remained fully intact after exposure to DPP IV for up to 24 hours. This implies that acylation of GIP with a C-16 palmitoyl group at position 16 or 37 masks the potential cleavage site for DPP IV. This was unexpected given that the 3-dimensional structure of GIP amounts to gentle helical turns around a strong backbone as revealed by computer-assisted secondary structure analysis (Alana, I. et al., 2004, Biochem. Biophys. Res. Commun. 325: 281-286).

Structural modification affecting the DPP IV cleavage site on the amino terminus of GIP is highly influential for bioactivity, potentially resulting in analogues with unchanged, increased, decreased or even antagonistic potency (Green, B. D. et al., 2004, Curr. Pharm. Des. 10: 3651-3662). Consistent with earlier studies, native GIP prominently stimulated cAMP production and insulin secretion in GIP receptor transfected CHL cells and BRIN-BD11 cells in vitro. Both GIP(LysPAL¹⁶), and GIP(LysPAL³⁷) exhibited similar dose-dependent effects to native GIP. This indicates that the fatty acid derivatised DPP IV resistant GIP molecules still retained full affinity for the GIP receptor and ability to activate signal transduction pathways culminating in stimulation of adenylate cyclase and insulin secretion. Thus, the inadvertent introduction of DPP IV resistance, by attachment of a pilmitoyl group at a Lysine, in particular at either Lys¹⁶ or Lys³⁷ of GIP, was not accompanied by compromised receptor activation and therefore represents a significant and unexpected attribute in terms of likely in vivo bioactivity.

Consistent with this view, GIP(LysPAL¹⁶) and GIP(LysPAL³⁷) displayed significantly enhanced antihyperglycaemic and insulin releasing activity when administered acutely with glucose to the commonly employed ob/ob mouse model (Bailey, C. J. and Flatt, P. R., 1982, Int. J. Obesity 6:11-21). Thus individual plasma glucose and insulin concentrations together with AUC measures were significantly improved compared with native GIP. The insulin response to GIP(LysPAL¹⁶), and especially GIP(LysPAL³⁷), were particularly prominent during the latter stages of the test. This is consistent with the ability of GIP analogues to overcome age-related deterioration of pancreatic beta cell glucose sensitivity. It also suggests that the two fatty acid derivatised analogues have a protracted in vivo half-life compared to native GIP. This is likely due to their inherent resistance to DPP IV degradation and also their affinity to bind to serum albumin, thus preventing kidney catabolism and subsequent removal from the body.

In support of protracted biological activity, single injection of the more potent agonist, GIP(LysPAL³⁷), resulted in a notable decline of plasma glucose that was sustained for at least 24 hours. This was associated with a trend towards elevated insulin, but concentrations were not significantly raised at any time point. This presumably reflects the glucose-dependent insulinotropic actions of GIP, although involvement of the established extrapancreatic actions of GIP cannot be totally discounted. In contrast, similar injection of native GIP to non-fasted ob/ob mice had little metabolic consequence even at 4 hours reflecting its rapid degradation by the ubiquitous enzyme DPP IV.

In a further part of this study, we examined the ability of extended daily treatment with GIP(LysPAL³⁷), N-AcGIP(LysPAL³⁷) and N-AcGIP to reverse aspects of the metabolic dysfunctions associated with these severely diabetic animals. Previous studies have already indicated the potential of the two latter DPP IV-resistant analogues of GIP to augment the glucose-dependent insulinotropic response and curtail the glycaemic excursion in ob/ob mice (O'Harte, F. P. M. et al., 2002, Diabetologia 45:1281-1291; Irwin, N. et al. 2005, J. Med. Chem. 48:1244-1250; Irwin, N. et al., 2005, J. Pharm. Expt. Therap. (in press)). The extended pharmacodynamic properties of N-AcGIP, N-AcGIP(LysPAL³⁷) and GIP(LysPAL³⁷) were confirmed in ob/ob mice, with significant beneficial effects observed on glucose tolerance 4 hours after administration.

Daily administration of N-AcGIP, N-AcGIP(LysPAL³⁷) and GIP(LysPAL³⁷) to ob/ob mice by intraperitoneal injection (25 nmoles/kg) resulted in significantly reduced plasma glucose levels in all three groups on day 14 when compared to controls. It is important to note that the aforementioned effects were witnessed independent of alterations in food intake or body weight. This accords with the view that GIP lacks effects on feeding activity. This also indicates that prolonged elevated plasma concentrations of GIP have no effect on gastric emptying (Meier, J. J. et al., 2003, Am. J. Phys. Endocrinol. Metab. 286:E621-625) and unlike the sister incretin GLP-1, does not lead to unwanted side-effects such as nausea and vomiting. Furthermore, serum glucagon levels were unchanged in the three groups on the final day of experimentation indicating that the beneficial effects of the agonists were not mediated through inhibition of glucagon secretion.

As expected, a significant part of the beneficial effect of GIP agonists in ob/ob mice resulted from potent insulinotropic actions. Although native GIP has been demonstrated as only a weak stimulus to insulin secretion in ob/ob mice at the age studied (O'Harte, F. P. M. et al., 2002, Diabetologia 45:1281-1291), daily administration of all three longer acting GIP molecules promoted an increased insulin response following beta-cell stimulation. This is consistent with the action of GIP as a promoter of proinsulin gene expression (Wang, Y. et al., 1996, Mol. Cell. Endocrinol. 116:81-87). These actions were highlighted in N-AcGIP(LysPAL³⁷) treated mice which displayed significantly elevated pancreatic insulin levels compared to control mice. Furthermore, the insulin response to glucose, native GIP and nutrient stimulation was significantly enhanced in ob/ob mice receiving N-AcGIP, N-AcGIP(LysPAL³⁷) and GIP(LysPAL³⁷). Notably, the enhanced insulin response resulted in reductions in glycaemic excursion following both intraperitonal glucose and feeding. Long-term treatment with all three analogues also modestly improved insulin sensitivity, indicating that improvement of the glucose lowering actions of secreted insulin also contributed to these effects. One major problem currently foreseen with extended GIP treatment is desensitization of hormone receptor action. There was no evidence that treatment with N-AcGIP, N-AcGIP(LysPAL³⁷) or GIP(LysPAL³⁷) for 14 days compromised the glucose lowering or insulin releasing actions of native GIP in any way. On the contrary, the effects of native GIP were markedly amplified in the three treatment groups when compared to controls on day 14. Thus, it appears that there may even be an improvement of pancreatic beta cell responsiveness to GIP.

The present study also indicates for the first time that each of the three analogues enhanced pancreatic beta cell mass by increasing the size and number of islets. This indicates that stable analogues of GIP may be useful for increasing endogenous beta cell mass and function in both type 1 and type 2 diabetes and in age-related deterioration of pancreatic beta cell glucose sensitivity. Since GIP may also protect against beta cell apoptosis, early treatment should also provide a means of protection against the beta cell demise that precedes diabetes onset. In support of positive effects on the genesis of beta cells in vivo, the present study showed clear effects of AcGIP(LysPAL¹⁶) on the differentiation of mouse embryonic stem cells into beta cell phenotype. This additionally suggests that stable GIP agonists may be useful in protocols designed to produce new insulin secreting cells from embryonic stem cells for cellular replacement therapy of diabetes.

Taken together the results of the present study indicate a marked improvement of diabetic state in ob/ob mice treated with the longer-acting GIP molecules. All three analogues, N-AcGIP, N-AcGIP(LysPAL³⁷) and GIP(LysPAL³⁷), possessed increased resistance to DPP IV degradation (O'Harte, F. P. M. et al., 2002, Diabetologia 45:1281-1291; Irwin, N. et al. 2005, J. Med. Chem. 48:1244-1250; Irwin, N. et al., 2005, J. Pharm. Expt. Therap. (in press)), which in view of the present findings appears sufficient to maximize their pharmacokinetic profile over an extended period. However, other avenues to extend the biological action of GIP analogues may offer additional advantages such as PEGylation, fusion of GIP with serum proteins or insertion of linker molecules that promote binding to circulating peptides in vivo. Another potentially useful future approach concerns the development of small non-peptidergic agonists of the GIP receptor.

In conclusion, these studies indicate that once daily injection of N-AcGIP, N-AcGIP(LysPAL³⁷) or GIP(LysPAL³⁷) to ob/ob mice for 14 days has significant anti diabetic effects and would be expected to counter age-related deterioration of pancreatic beta cell glucose sensitivity. It would appear these beneficial effects on glucose tolerance are mediated by promotion of insulin secretion with beneficial effects on the functional activity of pancreatic beta cells. Enhancement of insulin sensitivity and other extrapancreatic actions are also likely to be involved. Overall, these observations indicate that stable GIP agonists, such as N-AcGIP, N-AcGIP(LysPAL³⁷) and GIP(LysPAL³⁷) represent an important generation of future antidiabetic therapies. In addition to providing monotherapy, these agents may be used beneficially in combination therapy with other antidiabetic agents either targeting beta cells (namely, sulphonylureas, other insulin-releasing agents and GLP-1 receptor agonists) or extrapancreatic sites (such as metformin, thiazolidenediones, acarbose and guar gum).

### Bioavailability and Half-Life of Peptide Analogues

The facts that the peptide analogues significantly counter age-related deterioration of pancreatic beta cell glucose sensitivity by, for example, decreasing non-fasting plasma glucose, improving glucose tolerance, causing a significant enhancement in the insulin response to intraperitoneal glucose or nutrient intake, and improving insulin sensitivity and that N-AcGIP(LysPAL³⁷) can reverse the impaired insulin secretory response to glucose associated with aging, indicate the usefulness of the such peptide analogues, optionally as stable long-acting GIP agonists, including N-terminally protected PEGylated forms of GIP analogues.

PEG (polyethylene glycol) is a non-antigenic, water-soluble, biocompatible, inert polymer that significantly prolongs the circulatory half-life of a protein (Abuchowski, A. et al., 1984, Cancer Biochem. Biophys. 7:175-186; Hershfield, M. S. et al., 1987, N. Engl. J. Medicine 316:589-596; Meyers, F. J. et al., 1991, Clin. Pharmacol. Ther. 49:307-313), allowing the protein to be effective over a longer time. Covalent attachment of PEG (“PEGylation”) to a protein increases the protein's effective size and reduces its rate of clearance rate from the body. PEGylation can also result in reduced antigenicity and immunogenicity, improved solubility, resistance to proteolysis, improved bioavailability, reduced toxicity, improved stability, and easier formulation of peptides. Polyethylene glycol also does not aggregate, degrade or denature. Polyethylene glycol conjugates are thus stable and convenient for use in diagnostic assays. PEGs are commercially available in several sizes, allowing the circulating half-lives of PEG-modified proteins to be tailored for individual indications through use of different size PEGs.

One method for PEGylating proteins is to covalently attach PEG to cysteine residues using cysteine-reactive PEGs. A number of highly specific, cysteine-reactive PEGs with different reactive groups (e.g., maleimide, vinylsulfone) and different size PEGs (2-20 kDa) are commercially available (e.g., from Shearwater, Polymers, Inc., Huntsville, Ala., USA). At neutral pH, these PEG reagents selectively attach to “free” cysteine residues, i.e., cysteine residues not involved in disulfide bonds. The conjugates are hydrolytically stable. Use of cysteine-reactive PEGs allows the development of homogeneous PEG-protein conjugates of defined structure.

Native GIP(1-42) has no cysteines, however, considerable progress has been made in recent years in determining the structures of commercially important protein therapeutics and understanding how they interact with their protein targets, e.g., cell-surface receptors, proteases, etc. This structural information can be used to design PEG-protein conjugates using cysteine-reactive PEGs. Cysteine residues in most proteins participate in disulfide bonds and are not available for PEGylation using cysteine-reactive PEGs. Through in vitro mutagenesis using recombinant DNA techniques, additional cysteine residues can be introduced anywhere into the protein. The added cysteines can be introduced at the beginning of the protein, at the end of the protein, between two amino acids in the protein sequence or, preferably, substituted for an existing amino acid in the protein sequence. The newly added “free” cysteines can serve as sites for the specific attachment of a PEG molecule using cysteine-reactive PEGs. The added cysteine must be exposed on the protein's surface and accessible for PEGylation for this method to be successful. If the site used to introduce an added cysteine site is non-essential for biological activity, then the PEGylated protein will display essentially wild type (normal) in vitro bioactivity. When PEGylating proteins with cysteine-reactive PEGs, one should first identify the surface exposed, non-essential regions in the target protein where cysteine residues can be added or substituted for existing amino acids without loss of bioactivity.

Other approaches to extending the bioavailability of N-terminal analogues of GIP can also be employed by exploiting their binding to larger long lived proteins (Dennis, M. S. et al., 2002, J. Biol. Chem. 277:35035-35043). These approaches include genetic fusion with albumin or other plasma proteins, where the gene for GIP is fused with that of the larger protein (Osborn, B. L. et al., 2002, Eur. J. Pharmacol. 456:149-158).

Alternatively, drug affinity complex (DAC) technology (Holmes, D. L. et al., 2000, Bioconj. Chem. 11:439-444) can be employed whereby a short covalent chemical linker is introduced into the C-terminus of the N-terminally modified GIP molecule. This then interacts with a specific cysteine residue of circulating albumin to promote binding of the two molecules.

The binding of modified GIP to albumin or other large proteins can also be achieved by covalent linkage of the peptide to an antibody fragment that reacts with the longer lived protein in vivo.

Small molecular non-peptidergic activators or stimulators of the GIP receptor and associated signaling pathway can also be used. Thus, knowledge of the activity and 3-dimensional NMR structure of the bioactive domain of GIP (Alna, I. et al., 2004, Biochem. Biophys. Res. Comm. 325:281-286; Green, B. D. et al., 2004, Curr. Pharm. Design 10:3651-3662) would facilitate computer-aided ligand- and receptor-based drug design (Honma, T., 2003, Medic. Res. Rev. 23:606-632).

Alternatively, sceening of in silico databases containing non-peptide small molecules may be useful to identify prospective candidate GIP receptor mimetics and antagonists for biological testing (Alvarez, J. C., 2004, Curr. Opin. Chem. Biol. 8:365-370; Yoshimora, A. et al., 2005, Apoptosis 10:323-329).

These and similar alterations to extend the bioavailability and half-life of the peptide analogues are intended to be included in the invention.

The peptide analogues of the present invention have use in treating diseases and conditions associated with age-related decrease in pancreatic beta cell function. One or more of the peptide analogues can be used in a pharmaceutical composition, which can include a pharmaceutically acceptable carrier. Such a composition may also contain (in addition to the analogue and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration.

Administration of the peptide analogue of the present invention used in the pharmaceutical composition or to practice the method of the present invention can be carried out in a variety of conventional ways, such as by oral ingestion, inhalation, topical application or cutaneous, subcutaneous, intraperitoneal, parenteral or intravenous injection. Administration can be internal or external; or local, topical or systemic.

The compositions containing a peptide analogue of this invention can be administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier or vehicle.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

When a therapeutically effective amount of the composition of the present invention is administered orally, the composition of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the composition of the present invention, and preferably from about 1 to 50% of the composition of the present invention.

When a therapeutically effective amount of the composition of the present invention is administered by intravenous, cutaneous or subcutaneous injection, the composition of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to the composition of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

Use of timed release or sustained release delivery systems are also included. A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The therapeutic compositions can include pharmaceutically acceptable salts of the components therein, e.g., which may be derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al., describe pharmaceutically acceptable salts in detail in J. Pharmaceutical Sciences, 1977, 66:1 et seq., which is incorporated herein by reference in its entirety. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. The salts may be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptonoate, hexanoate, fumarate, hydrochloride, hydrobromide, hydroiodide, 2-hydroxymethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

As used herein, the terms “pharmaceutically acceptable”, “physiologically tolerable” and grammatical variations thereof as they refer to compositions, carriers, diluents and reagents, are used interchangeably and represent that the materials are capable of administration to or upon a mammal with a minimum of undesirable physiological effects such as nausea, dizziness, gastric upset and the like. The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The amount of peptide analogue of the present invention in the pharmaceutical composition of the present invention will depend upon the nature and severity of the condition being treated, on the nature of prior treatments which the patient has undergone, and on a variety of other factors, including the type of injury, the age, weight, sex, medical condition of the individual. Ultimately, the attending physician will decide the amount of the analogue with which to treat each individual patient. Initially, the attending physician will administer low doses of peptide analogue and observe the patient's response. Larger doses of peptide analogue may be administered until the optimal therapeutic effect is obtained for the patient, and at that point the dosage is not increased further.

Additional guidance on methods of determining dosages can be found in standard references, for example, Spilker, Guide to Clinical Studies and Developing Protocols, Raven Press Books, Ltd., New York, 1984, pp. 7-13 and 54-60; Spilker, Guide to Clinical Trials, Raven Press, Ltd., New York, 1991, pp. 93-101; Craig et al., Modern Pharmacology, 2d ed., Little Brown and Co., Boston, 1986, pp. 127-133; Speight, Avery's Drug Treatment: Principles and Practices of Clinical Pharmacology and Therapeutics, 3d ed., Williams and Wilkins, Baltimore, 1987, pp. 50-56; Tallarida et al., Principles in General Pharmacology, Springer-Verlag, New York, 1998, pp. 18-20; and Olson, Clinical Pharmacology Made Ridiculously Simple, MedMaster, Inc., Miami, 1993, pp. 1-5.

### Combination Therapies

Any of the peptide analogues as disclosed herein can be combined with antidiabetic treatments or agents having an anti diabetic effect. As used herein, “antidiabetic treatments” include agents which have an antidiabetic effect and agents which are used to treat or ameliorate diabetic symptoms. Such agents can include pharmaceutical agents such as, but not limited to, chemical, biochemical, peptide, peptidomimetic agents. Peptide agents can include one or more of the peptide analogues as disclosed herein, or other insulin-releasing hormones such as glucagon-like peptide-1 (GLP-1) or truncated or analogue versions of such peptides. The peptide analogues can also be combined with other treatment regimens such as dietary regimens.

As shown herein, the combination of a stable GIP agonist with an insulin sensitizer or other antidiabetic agents can be an effective means of countering the gradual development of glucose intolerance associated with aging. The present study shows that the antidiabetic potency of individual drugs can be enhanced by combining GIP analogues with antidiabetic drugs, such as sulphonylureas, meglitinide or insulin sensitizers. Various antidiabetic drugs are discussed below.

### Biguanides

Biguanides decrease glucose production by the liver. Metformin is a biguanide, and is marketed under the names “Glucophage” (metformin HCl tablets; Bristol-Myers Squibb Company) and “Glucophage XR” (metformin HCl extended release tablets; Bristol-Myers Squibb Company). Metformin also lowers total cholesterol, low density lipoproteins and triglycerides, and raises beneficial high density lipoproteins. Metformin is not generally used in patients with impaired liver or renal function, congestive heart failure, unstable heart disease, hypoxic lung disease, or advanced age.

### Sulphonylureas

Sulphonylurea medications work by increasing the amount of insulin made by the pancreas, and may be used for those patients who cannot take metformin. Possible side-effects include weight gain and hypoglycemia. Sulphonylureas include “first generation” sulfonylureas, which were marketed before 1984 (acetohexamide (Dymelor; Eli Lilly and Company), chlorpropamide (Diabinese; Pfizer Inc.), tolbutamide (Orinase; Pharmacia & Upjohn Inc.), tolazamide (Tolinase; Pharmacia & Upjohn Inc.)), and “second generation” sulfonylureas, which have been marketed since 1984 (glimepiride (Amaryl; Sanofi-Aventis S. A.), gliclazide (Diamicron; Servier), glipizide (Glucotrol, Glucotrol XL; Pfizer Inc.), glyburide, or glibenclamide (Diabeta; Aventis S. A.; Glynase PresTab, Micronase; Pharmacia & Upjohn Inc.)).

### Thiazolidinediones

Thiazolidinediones, also called glitazones, lower blood glucose by increasing insulin sensitivity, and can be taken in addition to metformin or a sulphonylurea. Thiazolidinediones include pioglitazone (e.g., pioglitazone hydrochloride (Actos; Takeda Chemicals Industries Ltd., Eli Lilly)), rosiglitazone (rosiglitazone maleate (Avandia; GlaxoSmithKline)), and troglitazone (Rezulin; Parke-Davis/Warner-Lambert).

### Meglitinides

Meglitinides stimulate insulin secretion of pancreatic beta cells, but are of a shorter duration than the sulphonyureas. Possible side-effects include weight gain and hypoglycemia. They can be administered alone or in combination with metformin. They include nateglinide (Starlix; Novartis Pharma AG) and repaglinide (Prandin, NovoNorm, or GlucoNorm; Novo Nordisk A/S).

### Alpha-Glucosidase Inhibitors

Alpha-glucosidase inhibitors delay the digestion of sugars and starches by delaying the absorption of carbohydrates from the gut, thereby reducing peaks of blood glucose which may occur after meals. Such inhibitors include acarbose (Precose, Prandase; Bayer North America), miglitol (Glyset, Diastabol; Pharmacia & Upjohn Inc., Sanofi).

### Incretin Mimetics

These drugs enhance glucose-dependent insulin secretion by pancreatic beta-cells, suppress inappropriately elevated glucagon secretion, and slow gastric emptying.

Exenatide (Byetta; Amylin Pharmaceuticals Inc.) is an incretin mimetic. It lowers blood glucose levels by increasing insulin secretion. It does this only in the presence of elevated blood glucose levels, and so tends not to increase the risk of hypoglycemia. Hypoglycemia can still occur if it is combined with a sulfonylurea, however. It is used to treat type 2 diabetes.

### Amylinomimetics

Pramlintide (e.g., pramlintide acetate; Symlin; Amylin Pharmaceuticals, Inc.) is a synthetic analogue of the hormone amylin, which is produced by pancreatic beta cells. Amylin, insulin and glucagon work together to maintain normal blood glucose levels. Pramlintide has been approved for patients with type 1 and type 2 diabetes. Pramlintide cannot be combined with insulin and must be injected separately.

### Other Antidiabetic Agents

Other agents, such as guar gum, can be used to slow intestinal glucose-absorption.

Agents to increase glucose excretion are also under development.

### Combination Drugs

Combination drugs are also available, such as those which combine metformin with another oral medication (e.g., glyburide and metformin HCl (Glucovance; Bristol-Myers Squibb Company), rosiglitazone maleate and metformin HCl (Avandamet; GlaxoSmithKline), and glipizide and metformin HCl (Metaglip; Bristol-Myers Squibb Company)).

### Other Peptide Sequences

In the treatments disclosed herein, it is preferable that analogues of human GIP are used. However, the GIP protein sequences from a number of animals are very similar to that of human, and these can also be used in the treatment methods disclosed herein. As used herein, the term a “peptide analogue of GIP” is intended to include other mammalian GIP polypeptide sequences which are similar to the human sequence and which can be used in the invention. The sequences for human (Moody et al. 1984 FEBS Lett. 172: 142-148), pig (Brown & Dryburgh 1971 Can. J. Biochem. 49: 867-872), cow (Carlquist et al. 1984 Eur. J. Biochem. 145: 573-577), hamster (Yasuda et al. 1994 Biochem. Biophys. Res. Commun. 205: 1556-1562), rat (Higashimoto et al. 1992 Biochim. Biophys. Acta 1132: 72-74) and mouse (Schieldrop et al. 1996 Biochim. Biophys. Acta 1308: 111-113) are provided below. The porcine GIP sequence differs from human at residues 18 and 34, while the bovine GIP sequence also differs at residue 37, etc. For all of the animal protein sequences, variations from the human primary sequence are capitalized and underlined.

(SEQ ID NO: 1)

| | |
|---|---|
| Human: | yaegtfisdysiamdkihqqdfvnwllaqkgkkndwkhnitq |

(SEQ ID NO:2)

| | |
|---|---|
| Pig: | yaegtfisdysiamdkiRqqdfvnwllaqkgkkSdwkhnitq |

(SEQ ID NO:3)

| | |
|---|---|
| Cow: | yaegtfisdysiamdkiRqqdfvnwllaqkgkkSdwIhnitq |

(SEQ ID NO:4)

| | |
|---|---|
| Hamster: | yaegtfisdysiamdkiRqqdfvnwllaqkgkkndwkhnitq |

(SEQ ID NO:5)

| | |
|---|---|
| Rat: | yaegtfisdysiamdkiRqqdfvnwllaqkgkkndwkhnLtq |

(SEQ ID NO:6)

| | |
|---|---|
| Mouse: | yeagtfisdysiamdkiRqqdfvnwllaqRgkkSdwkhnitq |

Species homologues of the disclosed proteins are also provided by the present invention. As used herein, a “species homologue” is a protein or polynucleotide with a different species of origin from that of a given protein or polynucleotide, but with significant sequence similarity to the given protein or polynucleotide. Preferably, polypeptide species homologues have at least 90% sequence identity, more preferably at least 95% identity most preferably at least 97% or 100% identity with the given polypeptide, where sequence identity is determined by comparing the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, primates, swine, cow, sheep, goat, hamster, rat, mouse, horse, or other species possessing GIP proteins of significant homology to that of human.

The invention also encompasses allelic variants of the disclosed GIP proteins; that is, naturally-occurring alternative forms of the GIP polypeptide which are identical or have significantly similar sequences to those disclosed herein. Preferably, allelic variants have at least 90% sequence identity, more preferably at least 95% identity most preferably at least 97% or 100% identity with the given polypeptide, where sequence identity is determined by comparing the amino acid sequences of the proteins when aligned so as to maximize overlap and identity while minimizing sequence gaps. Allelic variants may be isolated and identified by making suitable probes or primers from the sequences provided herein and screening a suitable nucleic acid source from individuals of the appropriate species.

The therapeutic compositions are also presently valuable for veterinary applications. Particularly domestic animals and thoroughbred horses, in addition to humans, are desired patients for such treatment with proteins of the present invention.

### Example 1

### In Vivo Use of Analogues of GIP to Treat Age-Related Glucose

These examples show the synthesis, purification and characterization of peptide analogues N-AcGIP, GIP(LysPAL¹⁶), GIP(LysPAL³⁷) and N-AcGIP(LysPAL³⁷), and methods for testing them in ob/ob mice. These examples also show methods for testing the analogue, N-AcGIP(LysPAL³⁷), for use in treating age-related glucose intolerance and insulin secretion.

### Synthesis, Purification and Characterization of Analogues of GIP

GIP analogues, N-AcGIP, GIP(LysPAL¹⁶), GIP(LysPAL³⁷) and N-AcGIP(LysPAL³⁷), and native GIP were sequentially synthesized on an Applied Biosystems automated peptide synthesizer (Model 432 A, Foster City, Calif., USA) using standard solid-phase Fmoc peptide chemistry as previously reported (O'Harte, F. P. M. et al., 2002, Diabetologia 45:1281-1291; Irwin, N. et al., 2005, Biol. Chem. (In Press)). In addition, an acetyl adduct was incorporated at the N-terminal Tyr¹ of the fatty acid derivatised or native GIP molecules where appropriate. N-AcGIP(LysPAL³⁷), GIP(LysPAL¹⁶) and GIP(LysPAL³⁷) were sequentially synthesized in the same way but with the exception that the lysine residue at position 16 or 37 was conjugated to an Fmoc protected C-16 palmitate fatty acid. The synthetic peptides were judged pure by reversed-phase HPLC on a Waters Millenium 2010 chromatography system (Software version 2.1.5) and subsequently characterized using matrix-assisted laser desorption ionization-time of flight (MALDI-TOF) mass spectrometry.

### Animals

Normal mice derived originally from the colony maintained at Aston University, UK (Flatt, P. R., and Bailey, C. J., 1981, Diabetologia 20:573-577) were used at 12-16 and 45-49 weeks of age. Obese diabetic (ob/ob) mice derived from the colony maintained at Aston University, UK (23) were used at 12-16 weeks of age. Animals were housed individually in an air-conditioned room at 22±2° C. with a 12 hours light: 12 hours dark cycle. Drinking water and standard rodent maintenance diet (Trouw Nutrition, Cheshire, UK) were freely available. All animal experiments were carried out in accordance with the UK Animals (Scientific Procedures) Act 1986. No adverse effects were observed following long-term administration of N-AcGIP, GIP(LysPAL³⁷) or N-AcGIP(LysPAL³⁷).

### Animal Experimental Procedures

Body weight and food intake were recorded over five days before commencement of metabolic tests. Intraperitoneal (i.p.) glucose tolerance (18 mmol/kg body weight) was assessed in non-fasted animals. Additional experiments were preformed in which native GIP and N-AcGIP(LysPAL³⁷) (25 nmol/kg bodyweight) were administered i.p. in combination with glucose. Blood samples, taken from the cut tip of the tail vein of conscious mice at times indicated in the Figures, were immediately centrifuged using a Beckman microcentrifuge (Beckman Instruments, High Wycombe, UK) for 30 seconds at 13,000 g. The resulting plasma was then aliquoted into fresh Eppendorf tubes and stored at −20° C. prior to glucose and insulin determinations.

Evaluation of the Effects of N-AcGIP, GIP(LysPAL¹⁶) GIP(LysPAL³⁷) and N-AcGIP(LysPAL³⁷) in ob/ob mice. Initially, the extended biological activity of N-AcGIP, GIP(LysPAL³⁷) and N-AcGIP(LysPAL³⁷) was examined in 18 hour fasted ob/ob mice in comparison with saline alone 4 hours after administration. Over a 14-day period, groups of ob/ob mice received once daily intraperitoneal injections (17:00 h) of either saline vehicle (0.9%, w/v, NaCl), N-AcGIP, GIP(LysPAL³⁷) and N-AcGIP(LysPAL³⁷) (all at 25 nmoles/kg body weight/day). Food intake and body weight were recorded daily from 5 days before commencement of the treatment regimes. Plasma glucose and insulin concentrations (10:00 h) were monitored at 2-4 day intervals. At 14 days, groups of animals were used to evaluate intraperitoneal glucose tolerance (18 mmoles/kg), insulin sensitivity (50 U/kg) and metabolic responses to native GIP (25 nmoles/kg). In a separate series, mice fasted for 18 hours were used to examine the metabolic response to 15 minutes feeding. All acute tests were commenced at 10:00 h. At the end of the 14-day treatment period, pancreatic tissues were excised for measurement of insulin following extraction with 5 ml/g ice-cold acid ethanol (75% ethanol, 2.35% H₂O, 1.5% HCl). Serum samples were taken for determination of glucagon concentrations. All other blood samples were collected from the cut tip of the tail vein of conscious mice into chilled fluoride/heparin coated glucose microcentrifuge tubes (Sarstedt, Nümbrecht, Germany) at the times indicated in the Figures. Blood samples were immediately centrifuged using a Beckman microcentrifuge (Beckman Instruments, Galway, Ireland) for 30 seconds at 13,000×g. The resulting plasma was then aliquoted into fresh tubes and stored at −20° C. prior to glucose and insulin determinations.

### Analyses

Plasma glucose was assayed by an automated glucose oxidase procedure using a Beckman glucose Analyser II (Stevens, J. F., 1971, Clin. Chem. Acta. 32:199-201). Plasma insulin was assayed by a modified dextran charcoal radioimmunoassay (Flatt, P. R., and Bailey, C. J., 1981, Diabetologia 20:573-577). In vivo data were compared using the unpaired Student's-t test. Area under the curve (AUC) analysis performed employed the trapezoidal rule (Burington, R. S., 1973, Handbook of Mathematical Tables and Formulae (New York: McGraw-Hill)). 1-5 Groups were considered to be significantly different if P<0.05.

### Results of In Vivo Metabolic Studies

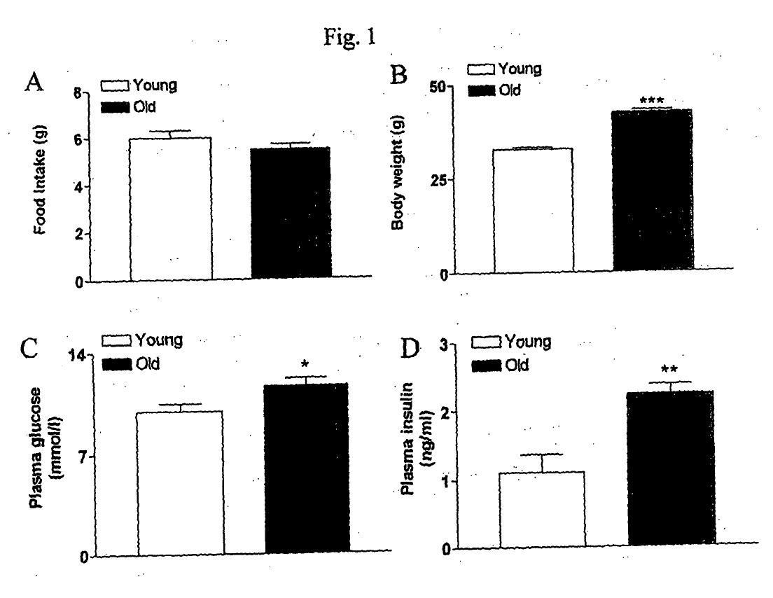

FIG. 1 shows the food intake (FIG. 1A), body weight (FIG. 1B), non-fasting plasma glucose (FIG. 1C) and insulin concentration (FIG. 1D) in younger (white bars) versus older (black bars) mice.

Food intake was not significantly different between younger and older mice, however body weights of the older mice were significantly (P<0.001) greater than the younger controls. Non-fasted plasma insulin concentrations were also significantly (P<0.01) elevated in older mice. Similarly basal plasma glucose concentrations were significantly (P<0.05) greater in older mice compared to younger controls.

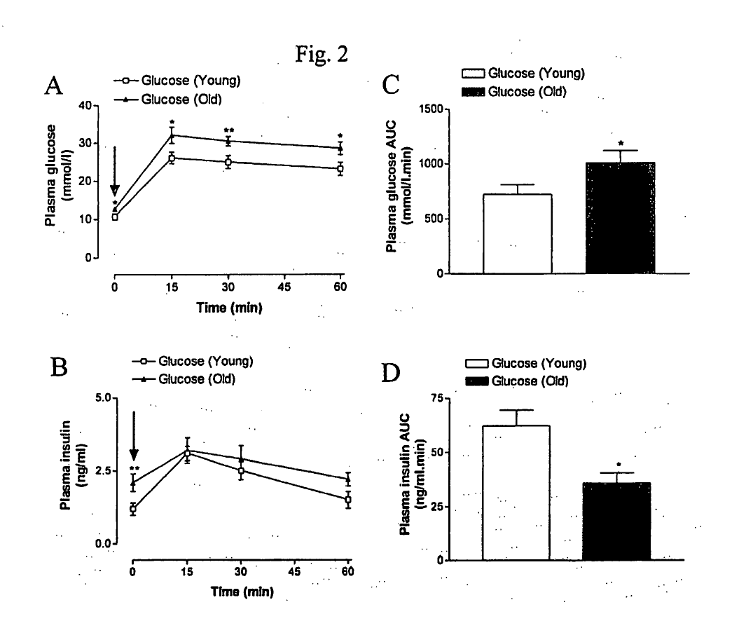

FIG. 2 presents the plasma glucose (FIGS. 2A, 2C) and insulin responses (FIGS. 2B, 2D) to an intraperitoneal glucose load in younger (□; white bars) versus older (▴; black bars) mice. Plasma glucose and insulin concentrations were significantly higher in older mice pre-injection (P<0.05 and P<0.01; respectively). The 0-60 min AUC values however, revealed a significantly decreased overall glucose-mediated insulin response (P<0.05) in older as compared to younger adult mice. Plasma glucose levels of older mice were also significantly elevated at 15, 30 and 60 min post glucose injection (P<0.05 to P<0.01) compared to younger controls. The overall glycaemic excursion of older mice was significantly (P<0.05) greater than that of younger control mice.

### Example 2

### In Vivo Effects of Native GIP and N-AcGIP(LysPAL³⁷) on Glucose Homeostasis and Insulin Secretion

This study examined the affects of a peptide analogue on glucose homeostasis and insulin secretion. N-AcGIP(LysPAL³⁷) and native GIP were synthesized as described above in Example 1, and animal experiments, procedures and analyses were likewise similar.

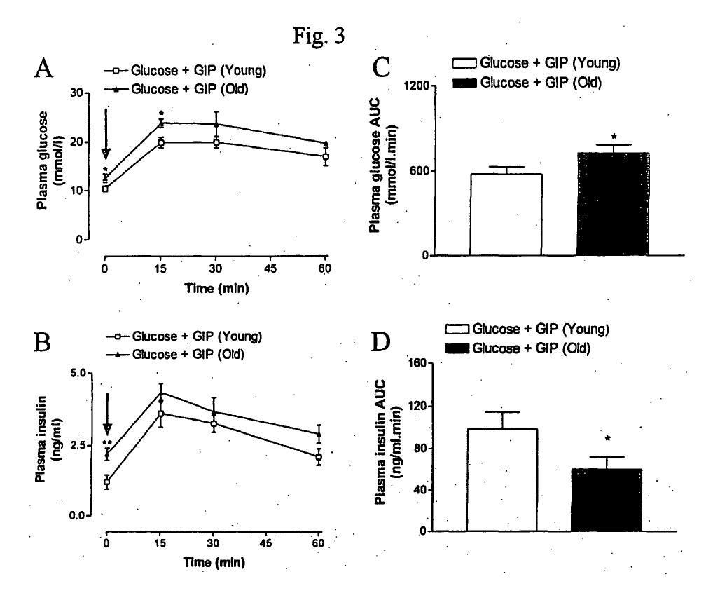

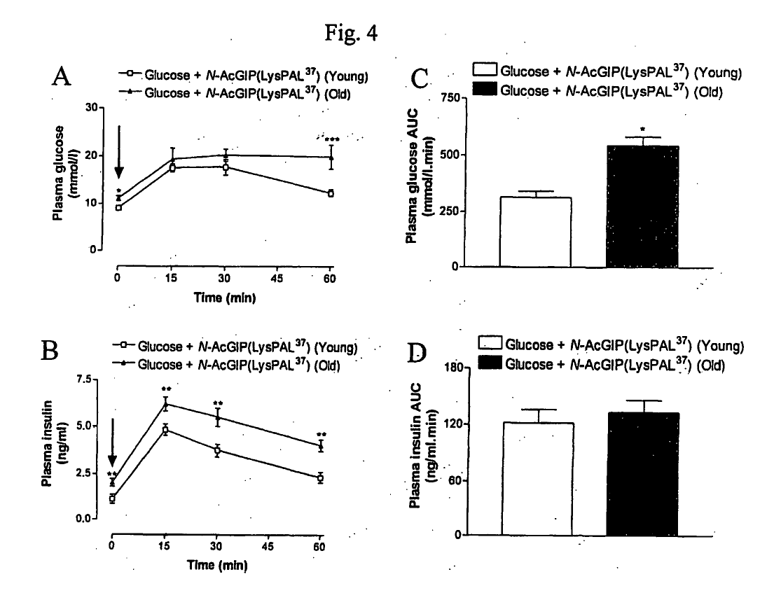

The results are shown in FIGS. 3 and 4, which display the effects of native GIP (FIG. 3) and N-AcGIP(LysPAL³⁷) (FIG. 4) on intraperitoneal glucose tolerance in older and younger control mice.

As with the response to glucose alone (FIG. 2), FIGS. 3A-3D show that the overall plasma glucose response (FIGS. 3A, 3C) to glucose plus native GIP was significantly elevated (P<0.05) in older (▴; black bars) mice compared to younger controls (o; white bars). Consistent with this, the overall plasma insulin response (FIGS. 3C, 3D) was significantly less in older mice (P<0.05).

As shown in FIGS. 4A-4D, N-AcGIP(LysPAL³⁷) restored the observed discrepancy in overall insulin release in older mice compared to younger controls (FIG. 4D). However, the overall glucose excursion was nonetheless significantly greater in older as opposed to younger mice (P<0.05) (FIG. 4C). Comparison of the two peptide treated groups revealed that N-AcGIP(LysPAL³⁷) resulted in significantly greater insulin response in both older and younger mice (P<0.01 to P<0.05). Both groups exhibited a significantly improved insulin response compared to mice receiving glucose alone (P<0.01 to P<0.00), whilst N-AcGIP(LysPAL³⁷) additionally reduced the overall glycaemic excursion compared to glucose alone in old and young mice (P<0.05).

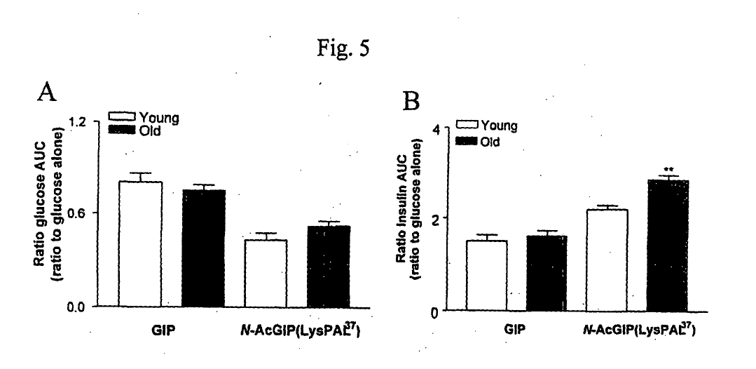

FIGS. 5A and 5B depict the ratio of the overall AUC hyperglycaemic (FIG. 5A) and insulinotropic (FIG. 5B) responses of young and old mice to native GIP and N-AcGIP(LysPAL³⁷) compared to glucose alone values. N-AcGIP(LysPAL³⁷) evoked a significantly enhanced insulin releasing action in older mice (P<0.01) compared to younger controls (FIG. 5B). No such effect for native GIP was observed. The overall glycaemic excursion induced by glucose in combination with either native GIP or N-AcGIP(LysPAL³⁷) were not significantly different in older and younger control mice. This is indicative of impaired insulin action in older adult mice.

### Example 3

### Effects of GIP in Combination With Other Antidiabetic Drugs In Vitro in BRIN-BD11 Cells

This Example studied the effects of GIP in combination with several known antidiabetic drugs in vitro in BRIN-BD11 cells.

### In Vitro Cellular Studies

Acute insulin-release studies were carried out using clonal pancreatic BRIN-BD11 cells, whose origin, characteristics and secretory responsiveness have been outlined in detail elsewhere (Irwin, N. et al., 2005, Biol. Chem. (In Press)). BRIN-BD11 cells were seeded into 24-multiwell plates at a density of 10⁵ cells per well and allowed to attach overnight at 37° C. Acute tests for insulin release were preceded by 40 min pre-incubation at 37° C. in 1.0 ml Krebs Ringer bicarbonate buffer (pH 7.4) supplemented with 0.5% (w/v) BSA and 1.1 mM glucose (estimated albumin concentration 76.5 μM). Test incubations were performed in the presence of 5.6 mM glucose with 10⁻⁸ M GIP and other antidiabetic agents as indicated in the Figures. After 20 minutes incubation, the buffer was removed from each well and aliquots (200 μl) were stored at −20° C. for measurement of insulin.

### Results

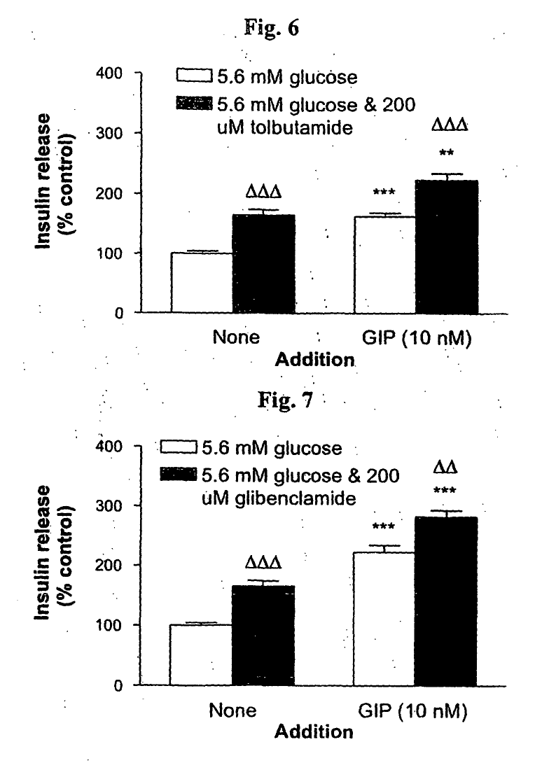

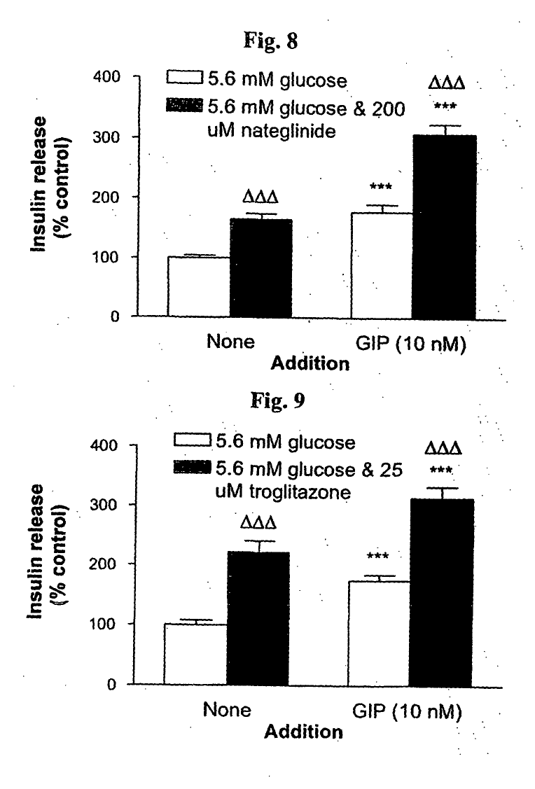

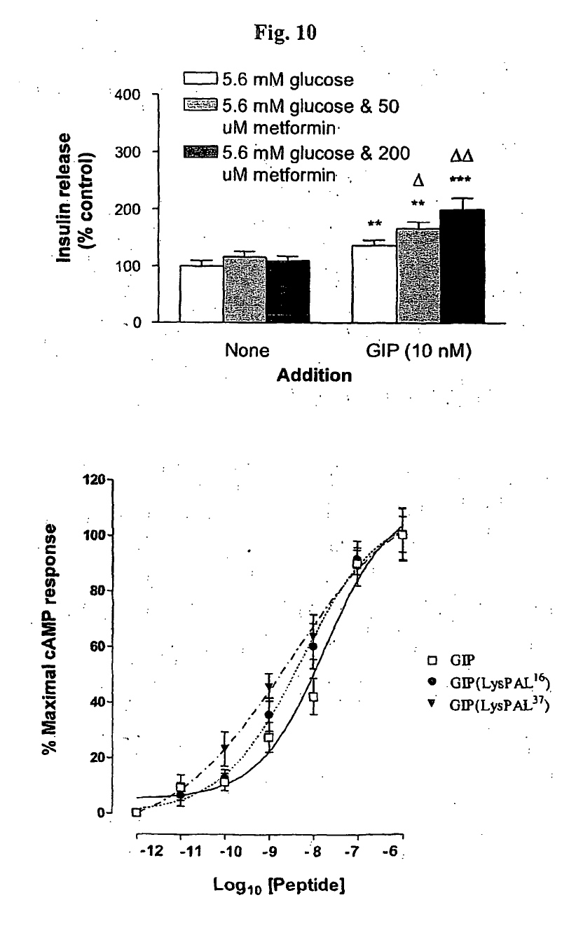

The results are shown in FIGS. 6-10. These figures show the effects on insulin secretion of GIP alone and in combination with sulphonylureas (tolbutamide (FIG. 6), glibenclamide (FIG. 7)), meglitinide (nateglinide) (FIG. 8), thiazolidenedione (troglitazone) (FIG. 9) and metformin (FIG. 10). GIP and other agents tested, except metformin, stimulated insulin secretion from BRIN-BD11 cells. Most notably, combination of GIP with tolbutamide, glibenclamide, nageglinide and both insulin sensitizers resulted in a significant potentiation compared with either agent alone.

### Example 4

### Degradation of Native GIP, GIP(LysPAL¹⁶) and GIP(LysPAL³⁷) By DPP IV

This study looked at the percentage intact peptide remaining after incubation with DPP IV.

### DPP IV Degradation Studies Using GIP(LysPAL¹⁶) and GIP(LysPAL³⁷).

Degradation of GIP and fatty acid derivatised GIP analogues were performed using HPLC. Briefly, GIP or GIP analogues were incubated in vitro at 37° C. in 50 mM triethanolamine-HCl (pH 7.8, final peptide concentration 2 mM) with purified porcine dipeptidyl peptidase IV (5 mU) for 0, 2, 8 and 24 hours. The reactions were subsequently terminated by addition of 10% (v/v) TFA/water and intact GIP was separated from the major degradation product GIP(342) by HPLC using a Vydac C-4 column (4:6×250 mm; The Separations Group, Hesparia, Calif., USA). Absorbance was monitored at 206 nm using a SpectraSystem UV 2000 Detector (Thermoquest Ltd., Manchester, UK) and the peaks collected manually prior to MALDI-TOF mass spectrometry. HPLC peak area data were used to calculate the percentage intact peptide remaining at the various time points during the incubation.

The results are shown in Table 1, below.

TABLE 1
Percentage intact peptide remaining after incubation with DPP IV.

| | % Intact peptide remaining after time (h) | | | |
|---|---|---|---|---|
| Peptide | 0 | 2 | 8 | 24 |
| Native GIP | 100 | 52 ± 3 | 0 | 0 |
| GIP(LysPAL¹⁶) | 100 | 100 | 100 | 100 |
| GIP(LysPAL³⁷) | 100 | 100 | 100 | 100 |

Values represent the % intact peptide remaining relative to the major degradation product GIP(3-42) following incubation with DPP IV as determined from HPLC peak area data. The reactions were performed in triplicate and the means ± SEM values calculated.

Table 1 shows that native GIP was rapidly degraded by DPP IV with only 52±3% of the peptide remaining intact after 2 hours of incubation. After 8 hours, GIP was completely degraded to GIP(3-42). In contrast, GIP(LysPAL¹⁶) and GIP(LysPAL³⁷) remained fully intact after prolonged incubations of up to 24 hours.

### Example 5

### Stimulation of cAMP by Native GIP, GIP(LysPAL¹⁶) and GIP(LysPAL³⁷)

This example examined intracellular cAMP production by GIP and GIP peptide analogues GIP(LysPAL¹⁶) and GIP(LysPAL³⁷) in vitro in CHL cells stably expressing the human GIP receptor. The results are shown in FIG. 11. Intracellular cAMP production was measured using GIP-receptor transfected Chinese hamster lung (CHL) fibroblasts. CHL cells were seeded into 12-multiwell plates (Nünc, Roskilde, Denmark) at a density of 10⁵ cells per well and allowed to grow for 48 hours before being loaded with tritiated adenine (2 μCi).

The cells were then incubated at 37° C. for 6 hours in 1 ml DMEM, supplemented with 0.5% (w/v) BSA and subsequently washed twice with HBS buffer (Hanks Buffered Saline solution). The cells were then exposed to varying concentrations of GIP/GIP analogues (10⁻¹³ to 10⁻⁶ M) in HBS buffer (estimated albumin concentration 76.5 μM) in the presence of 1 mM 3-isobutyl-1-methylxanthine (IBMX) for 15 minutes at 37° C. The medium was subsequently removed and the cells lysed with 1 ml of 5% trichloroacetic acid containing 0.1 mM unlabelled cAMP and 0.1 mM unlabelled ATP. The intracellular tritiated cAMP was then separated on Dowex and alumina exchange resins (Life Science Research, Larne, UK).

Acute insulin-release studies were carried out using clonal pancreatic BRIN-BD11 cells, whose origin, characteristics and secretory responsiveness have been outlined in detail elsewhere (McClenaghan, N. H. et al., 1996, Diabetes 45:1132-1140). BRIN-BD11 cells were seeded into 24-multiwell plates at a density of 10⁵ cells per well and allowed to attach overnight at 37° C. Acute tests for insulin release were preceded by 40 minutes pre-incubation at 37° C. in 1.0 ml Krebs Ringer bicarbonate buffer (pH 7.4) supplemented with 0.5% (w/v) BSA and 1.1 mM glucose (estimated albumin concentration 76.5 μM). Test incubations were performed in the presence of 5.6 mM glucose with a range of concentrations (10⁻¹³ to 10⁻⁶ M) of GIP or fatty acid derivatised GIP analogues. After 20 minutes incubation, the buffer was removed from each well and aliquots (200 μl) were stored at −20° C. for measurement of insulin.

Both GIP(LysPAL¹⁶) and GIP(LysPAL³⁷) stimulated cAMP production in a concentration-dependent-manner in GIP receptor transfected fibroblasts (FIG. 11). The calculated EC₅₀ values for GIP, GIP(LysPAL¹⁶) and GIP(LysPAL³⁷) were 18.2, 2.9 and 5.4 nmol, respectively, indicating both GIP analogues were slightly more potent than native GIP.

### Example 6

### Stimulation of In Vitro Insulin Secretion by Native GIP, GIP(LysPAL¹⁶) and GIP(LysPAL³⁷)

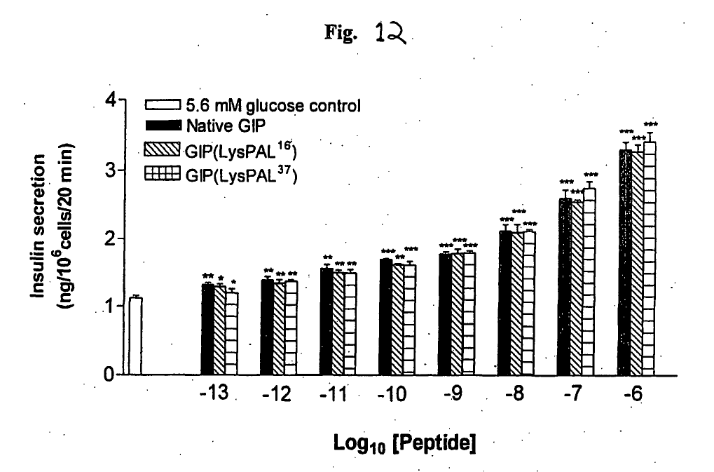

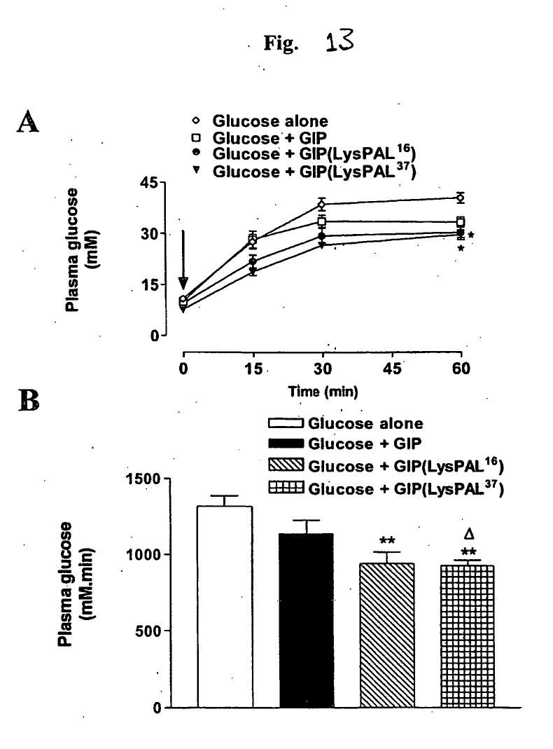

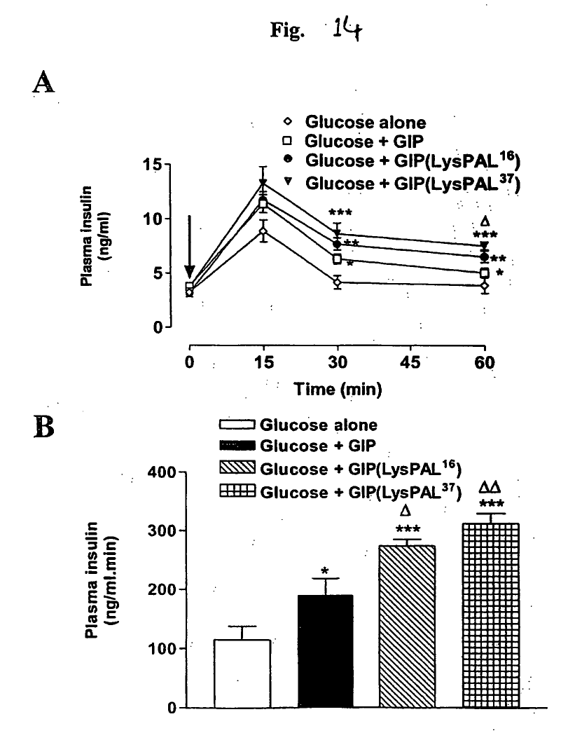

The insulin-releasing activity of GIP, GIP(LysPAL¹⁶) and GIP(LysPAL³⁷) in the clonal pancreatic beta cell line, BRIN-BD11, was also examined. The results are shown in FIG. 12.

Both GIP(LysPAL¹⁶) and GIP(LysPAL³⁷) enhanced insulin release in a concentration-dependent manner similar to native GIP (FIG. 12). Over the range of concentrations tested (10⁻¹³ to 10⁻⁶ M) insulin secretion was stimulated by 1.1- to 2.5-fold (P<0.05 to P<0.001) compared to 5.6 mM glucose control. There was no significant difference in potency between the three peptides.

### Example 7

### Acute In Vivo Effects of Native GIP, GIP(LysPAL¹⁶) and GIP(LysPAL³⁷)

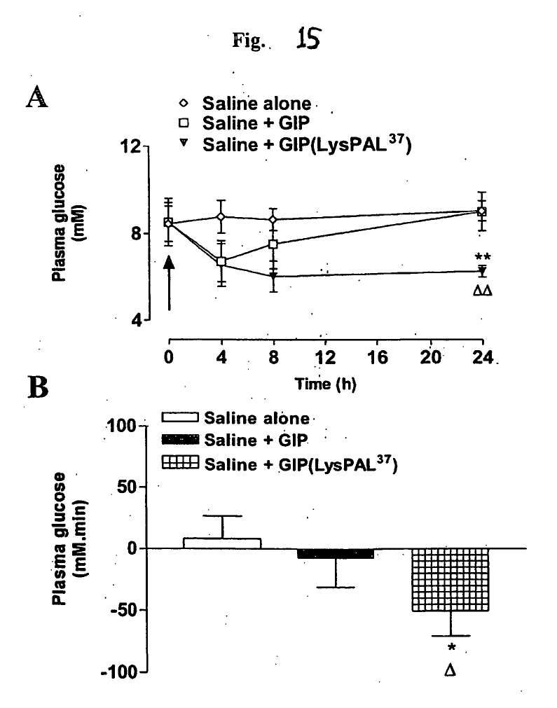

The glucose lowering effects and insulin releasing activity of GIP, GIP(LysPAL¹⁶) and GIP(LysPAL³⁷) in 18 hour fasted (ob/ob) mice were also studied. The results are shown in FIGS. 13A-13B and 14A-14B.

The relative glucose-lowering abilities of GIP, GIP(LysPAL¹⁶) or GIP(LysPAL³⁷) (25 mmol kg⁻¹ body weight) in ob/ob mice are shown in FIG. 13. Injection of glucose alone, resulted in a rapid and marked increase in plasma glucose which continued to rise until 60 minutes. Native GIP had a tendency to reduce glucose at the later time points monitored, but neither this nor the overall AUC glucose excursion achieved significance (FIGS. 13A, 13B). In contrast, GIP(LysPAL¹⁶) and GIP(LysPAL³⁷) significantly reduced plasma glucose concentrations from 15 to 60 minutes and AUC values when compared to glucose alone. GIP(LysPAL³⁷) further revealed a significant reduction in the AUC response compared to native GIP. As shown in FIG. 14, these effects were linked to corresponding changes in plasma insulin concentrations. GIP caused a significantly greater (P<0.05) insulin release than glucose alone. However, most notably GIP(LysPAL¹⁶) and GIP(LysPAL³⁷) exhibited substantially greater and more protracted insulin responses than native GIP. Thus plasma insulin from 30 to 60 minutes and AUC values were significantly increased (FIGS. 14A, 14B). The overall enhancement of insulin release by GIP(LysPAL¹⁶) and GIP(LysPAL³⁷) over native GIP was 2.4- and 2.7-fold (P<0.05 to P<0.01), respectively.

### Example 8

### Longer Term Actions of Native GIP and GIP(LysPAL³⁷) ob/ob Mice

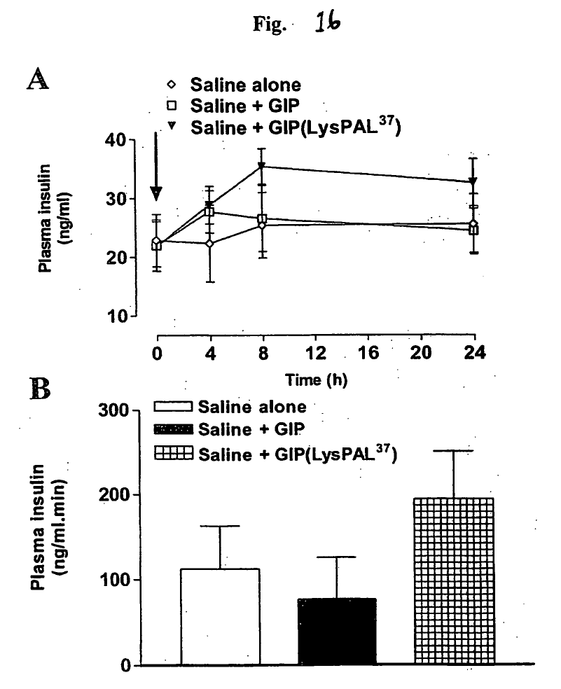

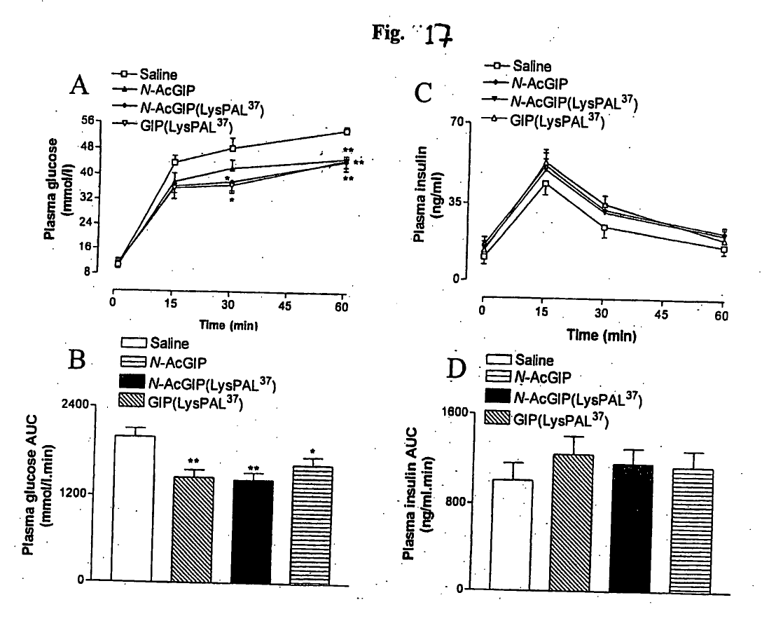

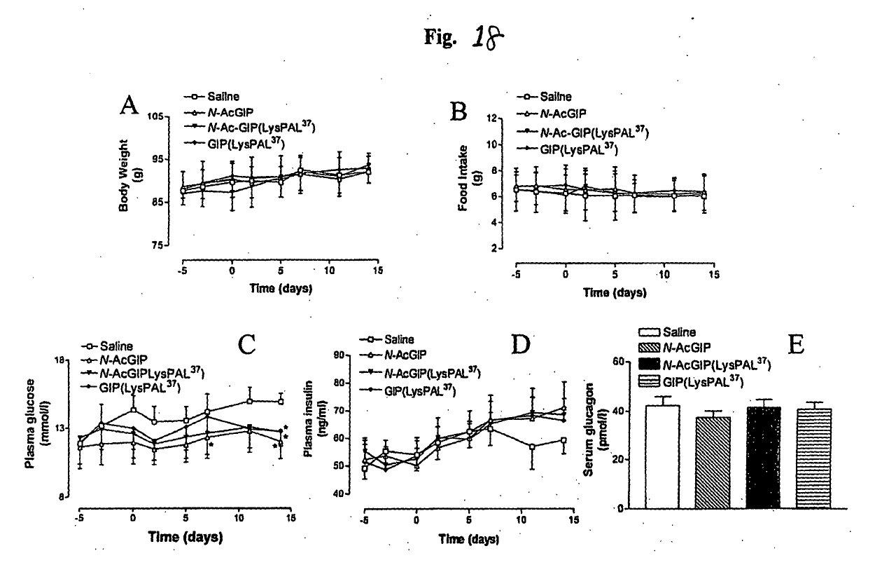

Native GIP and GIP(LysPAL³⁷) were also studied for their effects on prolonging the glucose lowering effects and insulinotropic effects in ob/ob mice. The results are shown in FIGS. 15A-15B and 16A-16B.

Dose of 12.5 nmol kg⁻¹ was chosen to evaluate the longer-term duration of action of a single dose of native or fatty acid derivatised GIP in non-fasted ob/ob mice. As shown in FIG. 15, GIP had a transient, but not significant effect on plasma glucose. In contrast, GIP(LysPAL³⁷) induced a significant (P<0.01) and sustained decrease of glycaemia. Glucose concentrations at 24 hours and AUC values were significantly less (P<0.05 to P<0.01) than native GIP or saline treated controls. Insulin concentrations had a tendency to be higher in the GIP(LysPAL³⁷) group from 4 to 24 hours, but values were not significantly different from either groups of controls (FIG. 16).

### Example 9

### Effects of N-AcGIP, GIP(LysPAL³⁷) and N-AcGIP(LysPAL³⁷) on Plasma Glucose and Insulin Concentrations 4 Hours after Administration

The effects of N-AcGIP, N-AcGIP(LysPAL³⁷) and GIP(LysPAL³⁷) on plasma glucose and insulin response 4 hours after administration were examined. The results are shown in FIGS. 17A-17D.

As shown in FIG. 17, administration of N-AcGIP, N-AcGIP(LysPAL³⁷) and GIP(LysPAL³⁷) decreased the glycaemic excursion and glucose levels following i.p. glucose injection (I 8 mmoles/kg body weight) 4 hours after administration in 18 hour fasted ob/ob mice (27% reduction; p<0.01, 28% reduction; p<0.01, 18% reduction; p<0.05; respectively). This supports a protracted biological half-life of N-AcGIP, N-AcGIP(LysPAL³⁷) and GIP(LysPAL³⁷) and forms the basis of the once-daily injections. In comparison, the insulinotropic properties of N-AcGIP, N-AcGIP(LysPAL³⁷) and GIP(LysPAL³⁷) were less evident 4 hours after administration, supporting the idea that the extrapancreatic actions of the GIP analogues are important for their glucose lowering actions.

### Example 10

### Effects of N-AcGIP, N-AcGIP(LysPAL³⁷) and GIP(LysPAL³⁷) on Food Intake, Body Weight and Non-Fasting Plasma Glucose, Glucagon and Insulin Concentrations

FIGS. 18A-18E show the effects of daily N-AcGIP, N-AcGIP(LysPAL³⁷) and GIP(LysPAL³⁷) administration on food intake (FIG. 18A), body weight (FIG. 18B), plasma glucose (FIG. 18C), insulin (FIG. 18D) and final glucagon levels (FIG. 18E). Administration of N-AcGIP, N-AcGIP(LysPAL³⁷) or GIP(LysPAL³⁷) had no effect on food intake or body weight (FIG. 18). Plasma glucose concentrations of N-AcGIP, N-AcGIP(LysPAL³⁷) and GIP(LysPAL³⁷) treated mice displayed a progressive reduction, resulting in significantly (p<0.05) lowered glucose concentrations at 14 days in all three treatment groups (FIG. 18). These changes were accompanied by a tendency towards elevated insulin concentrations, but these did not achieve statistical significance over the time frame studied (FIG. 18). On day 14, plasma glucagon was also assessed with no differences observed between control and treatment groups (FIG. 18).

### Example 11

### Effects of N-AcGIP, N-AcGIP(LysPAL³⁷) and GIP(LysPAL³⁷) on Glucose Tolerance

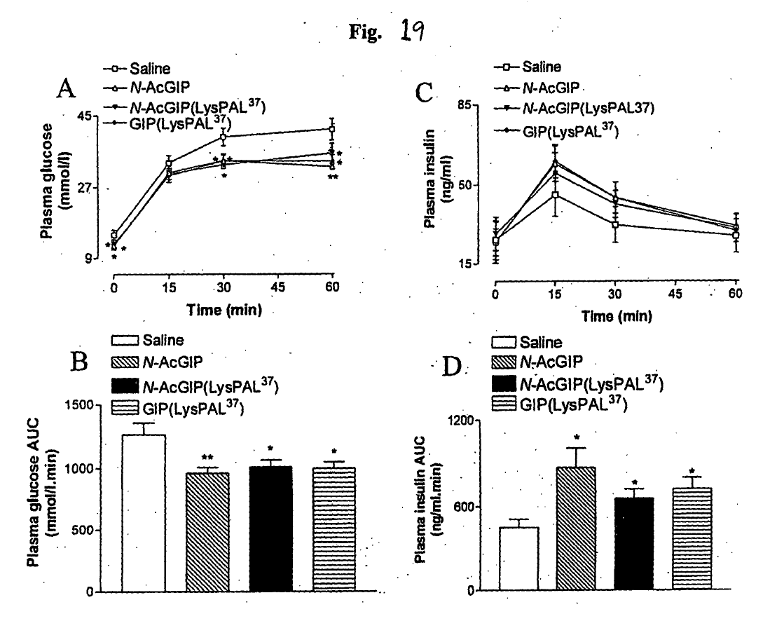

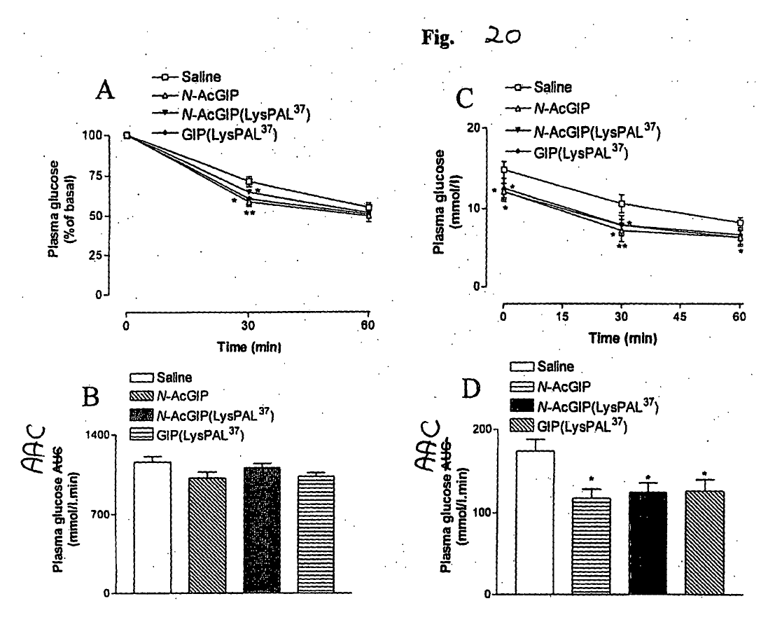

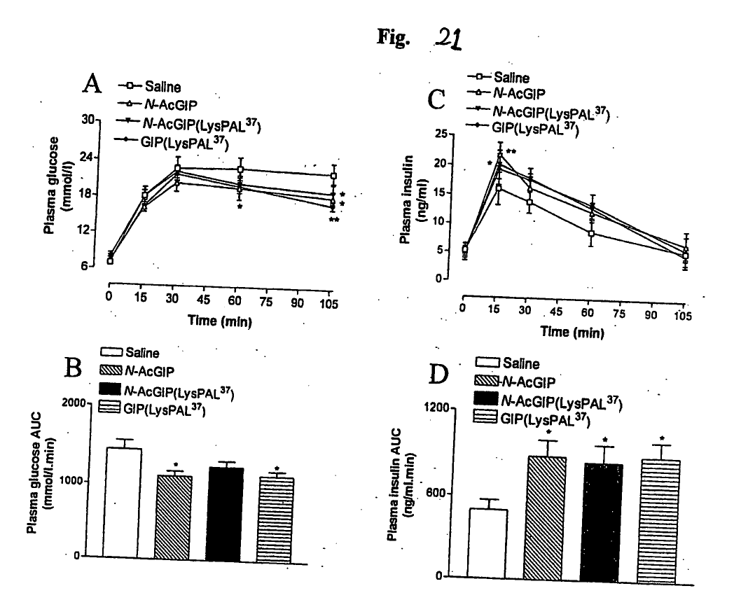

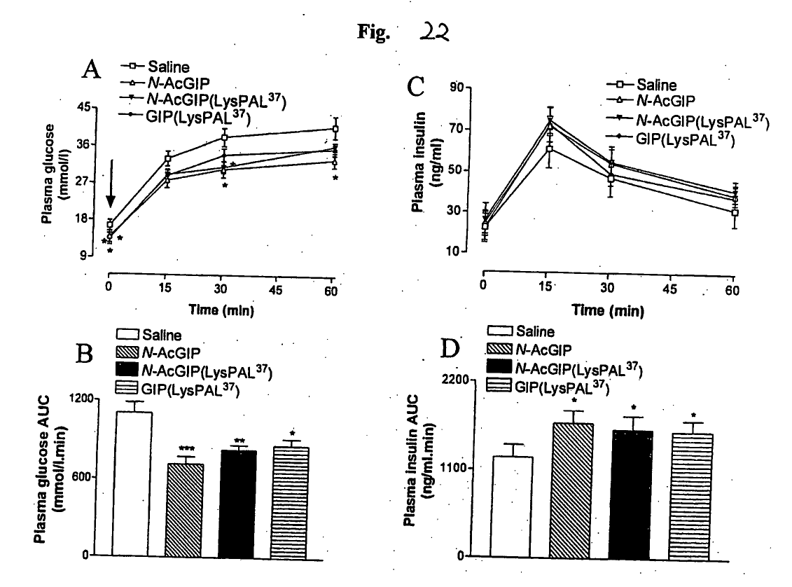

The effects of daily N-AcGIP, N-AcGIP(LysPAL³⁷) and GIP(LysPAL³⁷) administration on glucose tolerance (FIGS. 19A, 19B) and plasma insulin response to glucose (FIGS. 19C, 19D) were also studied.

Treatment of ob/ob mice for 14 days with N-AcGIP (Δ, diagonally cross-hatched bars), N-AcGIP(LysPAL³⁷) (▾, black bars), or GIP(LysPAL³⁷) (♦, horizontally cross-hatched bars) resulted in a significant improvement in glucose tolerance, relative to saline-treated controls (□, white bars). N-AcGIP produced a distinct reduction in the overall glycaemic excursion (24% reduction; p<0.01) (FIGS. 19A, 19B). This was accompanied by an overall increased insulin response (137% increase; p<0.05) (FIG. 19D). Similarly both fatty acid derivatised GIP analogues significantly reduced the overall glycaemic excursion (FIGS. 19A, 19B) and augmented the insulinotropic response following i.p. glucose administration (FIG. 19D) (p<0.05; in both cases).

### Example 12

### Effects of N-AcGIP, N-AcGIP(LysPAL³⁷) and GIP(LysPAL³⁷) on Insulin Sensitivity

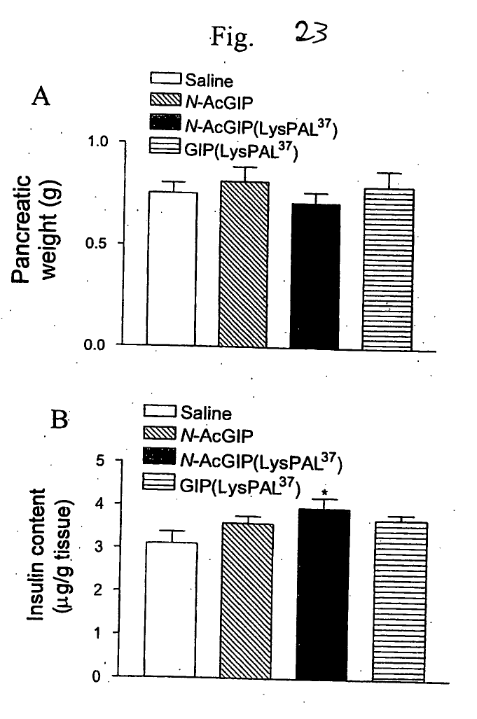

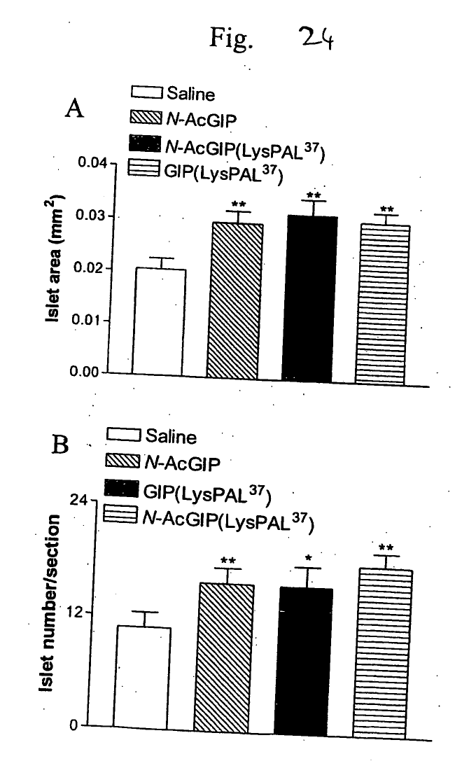

This example studies the effects of daily N-AcGIP, N-AcGIP(LysPAL³⁷) and GIP(LysPAL³⁷) administration on insulin sensitivity. The results are shown in FIGS. 20A-20D.

As shown in FIG. 20, the hypoglycemic action of insulin was significantly augmented in terms of AUC measures (p<0.05) and post injection values (p<0.05 to p<0.01) in ob/ob mice treated with N-AcGIP, N-AcGIP(LysPAL³⁷) or GIP(LysPAL³⁷) for 14 days when compared to controls.

### Example 13

### Effects of N-AcGIP, N-AcGIP(LysPAL³⁷) and GIP(LysPAL³⁷) on Metabolic Response to Feeding

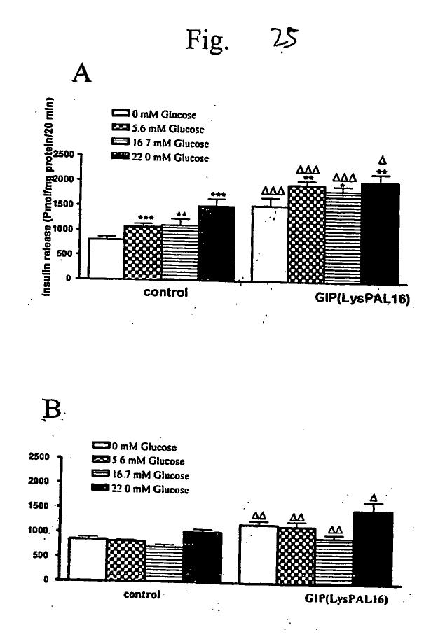

The effects of daily N-AcGIP, N-AcGIP(LysPAL³⁷) and GIP(LysPAL³⁷) administration on glucose (FIGS. 21A, 21B) and insulin (FIGS. 21C, 21D) responses to feeding in 18 hours fasted ob/ob mice were examined.

Plasma glucose responses to 15 minutes feeding were significantly lowered (p<0.05) at 15 minutes in ob/ob mice treated with N-AcGIP, N-AcGIP(LysPAL³⁷) or GIP(LysPAL³⁷) for 14 days (FIGS. 21A, 21B). Furthermore, the overall glycaemic response was significantly reduced in N-AcGIP treated mice (p<0.05). Similarly, plasma insulin responses to 15 minutes feeding were significantly increased (p<0.05) at 15 minutes in ob/ob mice treated with N-AcGIP, N-AcGIP(LysPAL³⁷) or GIP(LysPAL³⁷) for 14 days, with an overall increased insulinotropic response observed in all three treatment groups mice (FIGS. 21C, 21D).

### Example 14

### Effects of N-AcGIP, N-AcGIP(LysPAL³⁷) and GIP(LysPAL³⁷) on Response to Native GIP

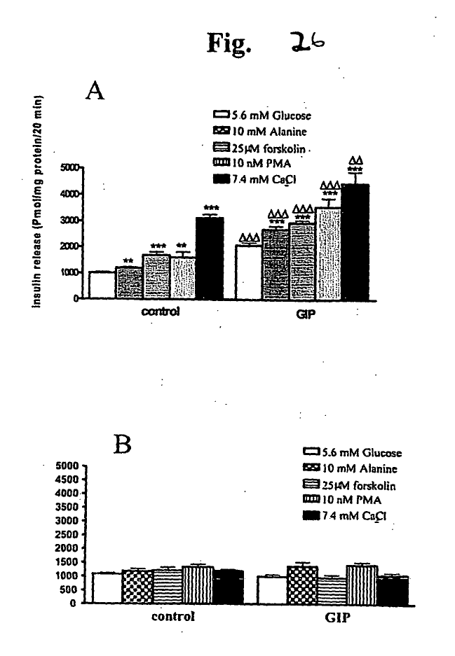

Effects of daily N-AcGIP, N-AcGIP(LysPAL³⁷) and GIP(LysPAL³⁷) administration on glucose tolerance and plasma insulin response to native GIP were studied, and the results are shown in FIGS. 22A-22D.

GIP receptor desensitization was assessed thorough changes in the metabolic responses to native GIP in combination with glucose in 14 day treated N-AcGIP (Δ, diagonally cross-hatched bars), N-AcGIP(LysPAL³⁷) (▾, black bars) and GIP(LysPAL³⁷) (♦, horizontally cross-hatched bars) mice. As shown in FIGS. 22A-D, treatment of ob/ob mice with N-AcGIP, N-AcGIP(LysPAL³⁷) or GIP(LysPAL³⁷) for 14 days significantly augmented the overall glucose-dependent insulinotropic response to native GIP (p<0.05, in all cases). In harmony with this 14-day treated N-AcGIP, N-AcGIP(LysPAL³⁷) or GIP(LysPAL³⁷) mice displayed significantly decreased glycaemic excursions compared to control animals (p<0.01, p<0.01, p<0.05; respectively). Thus 14-day treatment with N-AcGIP, N-AcGIP(LysPAL³⁷) and GIP(LysPAL³⁷) augmented the insulinotropic and glucose homeostatic actions of native GIP in ob/ob mice.

### Example 15

### Effects of N-AcGIP, N-AcGIP(LysPAL³⁷) and GIP(LysPAL³⁷) on Pancreatic Insulin Content

This example shows the effects of daily N-AcGIP, N-AcGIP(LysPAL³⁷) and GIP(LysPAL³⁷) administration on pancreatic weight and insulin content. The results are shown in FIGS. 23A-23B. Treatment of ob/ob mice for 14 days with N-AcGIP (diagonally cross-hatched bars), N-AcGIP(LysPAL³7) (black bars) and GIP(LysPAL³⁷) (horizontally cross-hatched bars) did not affect pancreatic weight compared with saline-treated controls (FIG. 23A). However, daily administration of N-AcGIP(LysPAL³⁷) significantly increased (p<0.05) insulin content compared with controls (FIG. 23B). N-AcGIP and GIP(LysPAL³⁷) treatment did not affect pancreatic insulin content when compared with saline-treated controls.

### Example 16

### Effects of N-AcGIP, N-AcGIP(LysPAL³⁷) and GIP(LysPAL³⁷) on Islet Morphology

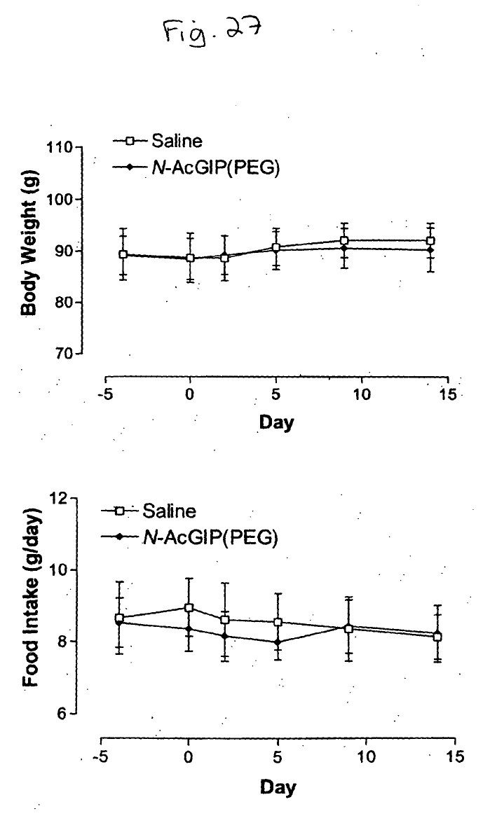

This example studied the effects of daily N-AcGIP, N-AcGIP(LysPAL³⁷) and GIP(LysPAL³⁷) administration on islet area and islet number. The results are shown in FIGS. 24A and 24B, which are a pair of bar graphs.

Treatment of ob/ob mice for 14 days with N-AcGIP (diagonally cross-hatched bars), N-AcGIP(LysPAL³7) (black bars) and GIP(LysPAL³⁷) (horizontally cross-hatched bars) resulted in a significant increases in both islet area (FIG. 24A) and number of pancreatic islets (FIG. 24B) compared with saline-treated ob/ob mice.

### Example 17

### Effects of GIP(LysPAL¹⁶) on Production of Insulin and C-Peptide from Differentiated D3 Cluster Cells

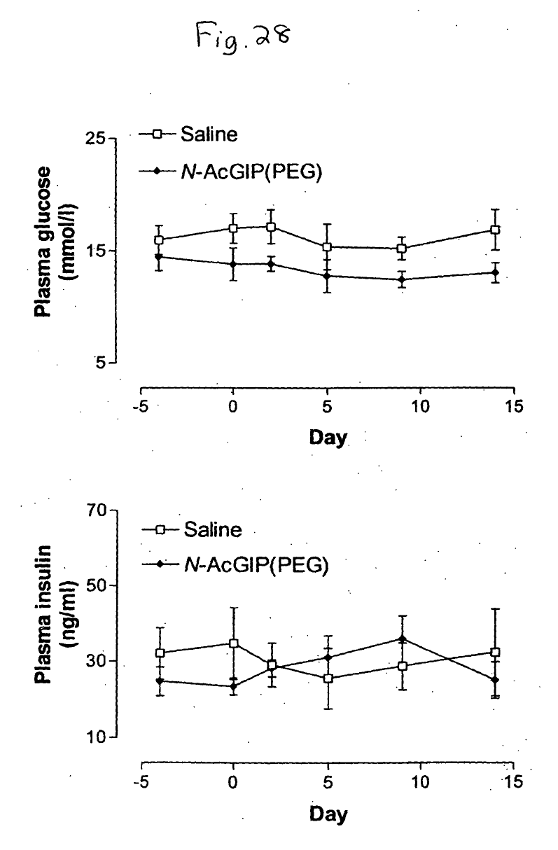

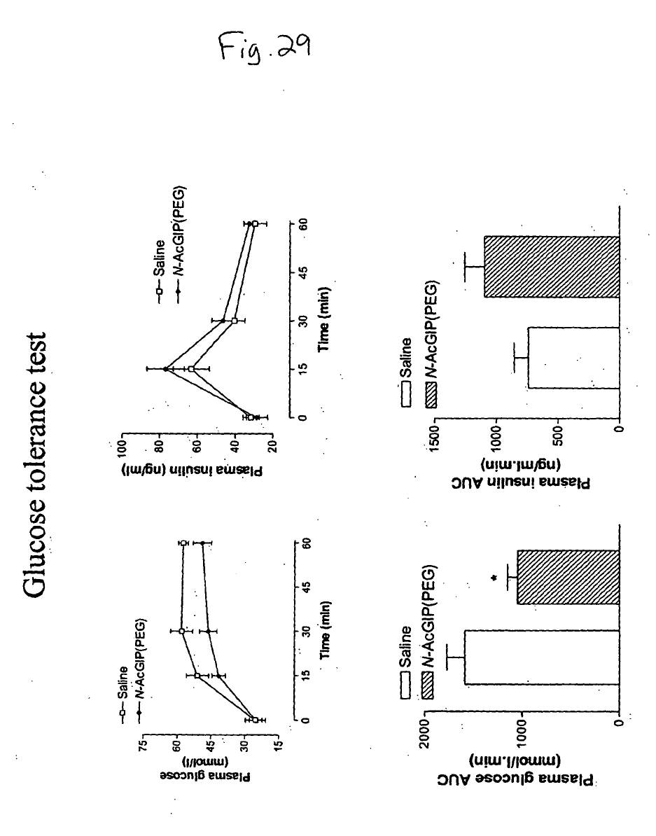

This example studied insulin secretion from differentiated D3 cells after exposure to different levels of glucose, and after exposure to various secretagogues. The results are shown in FIGS. 25A-25B and 26A-26B, respectively.

Evaluation of the Effects of GIP(LysPAL¹⁶) on differentiation of embryonic stem (ES) cell to beta cell phenotype. The D3 mouse ES cell line was routinely cultured on tissue culture plastic coated with 0.1% (w/v) gelatine in DMEM (25 mM glucose) containing 15% foetal bovine serum (FBS), 2 mM glutamine, 50 U/ml penicillin/streptomycin, non-essential amino acids, 0.1 mM β-mercaptoethanol, and 1000 U/ml LIF (leukaemia inhibitory factor). Medium was changed daily and cultures were passaged every 2 days onto fresh geletin-coated plates at a split ratio of 1:5. The cells were passaged by washing the cultures with phosphate-buffer saline and then detached from the plate with a trypsin (2.5 g/L)-EDTA (1 mM) solution. Prior to seeding into new flasks the ES cells were dissociated to single cells suspensions by repeated pipetting.

For differentiation of D3 mouse ES cells, an established protocol was used (Lumelsky, N. et al., 2001, Science 292(5520):1389-94). Briefly, LIF was removed from the culture medium, EBs (embryonic bodies) were then formed by growing the cells as non-adherent clusters. For the selection of nestin positive precursor cells, medium was changed to serum free ITSFn medium and for expanding these progenitors, cells were incubated in N2 serum free medium for 7 days (stage 4) and then differentiated into insulin-containing cell clusters by incubation in N2 serum free medium without bFGF, supplemented with 10 mM nicotinamide for 7 days (stage 5) (control protocol). During the differentiation process the protocol was modified by supplementation of stage 4 and 5 culture media with 1×10⁻⁶ M GIP(LysPAL¹⁶). The differentiated cells were harvested at the end of stage 4 and 5 and samples were seeded onto 24 well plates for insulin and C-peptide studies. Insulin release from differentiated cells from stages 4 and 5 were determined using cell monolayers. The cells were harvested with the aid of trypsin/EDTA (Gibco), seeded into 24-multiwell plates (Nunc, Rosklide, Denmark) at a density of 0.1×10⁶ cells per well, and allowed to attach overnight.

Prior to the acute test, cells were washed three times and preincubated for 40 minutes at 37° C. in a 1.0 ml Krebs Ringer bicarbonate buffer (115 mM NaCl, 4.7 mM KCl, 1.28 mM CaCl₂, 1.2 mM KH₂PO₄, 1.2 mM MgSO₄, 10 mM NaHCO₃, 5 g/l bovine serum albumin, pH 7.4) supplemented with 1.1 mM glucose. Test incubations were performed for 20 minutes at 37° C. using the same buffer supplemented with 5.6 mM glucose in the absence (control) and presence of various test agents as indicated in the Figures. After incubation, aliquots of buffer were removed and stored at −20° C. for insulin radioimmunoassay. For assessment of cellular hormone contents, the buffer was completely removed and 1000% of acid-ethanol solution (1.5% (v/v) HCl, 75% (v/v) ethanol, 23.5% (v/v) H₂O) was added. The cells were disrupted with the aid of a pasture pipette and incubated overnight at 4° C. prior to centrifugation (900 rpm) and stored at −20° C.

Glucose (FIGS. 25A and 25B) and secretagogue-induced (FIGS. 26A and 26B) insulin release were assessed using differentiated D3 cells at stages 4 (FIGS. 25A, 26A) and stage 5 (FIGS. 25B, 26B) after exposure to the various culture conditions.

The cells were treated with varying levels of glucose (FIGS. 25A and 25B). In the control protocol (left group in FIGS. 25A and 25B), the differentiated cells at stage 4 release insulin in response to glucose in a dose dependent manner (FIG. 25A). Similar effects were observed in cells cultured in media supplemented with 1×10⁻⁶ M GIP(LysPAL¹⁶) (right group in each figure). In addition, insulin releasing effects of the differentiated cells in the modified protocol incorporating GIP(LysPAL¹⁶) were significantly higher than in the control protocol. In contrast, differentiated stage 5 cells from both protocols appear not respond to glucose (FIG. 25B, left group).

To determine whether the differentiated cells use physiological pathways to regulate insulin release, the cells were tested with several insulin secretagogues. The results are shown in FIGS. 26A and 26B. Stage 4 cluster cells (FIG. 26A) in the control protocol (left group) responded with 1.2-3-fold increased insulin release with, 10 mM alanine, 25 μM forskolin, 10 nM PMA and 7.4 mM CaCl₂ compared with 5.6 mM glucose (P<0.001). The stage 4 cells from the modified protocol (FIG. 26A, right group) also showed similar effects to the secretagogues with the cells releasing higher insulin than those from the control protocol (FIG. 25). As with the response to glucose, the differentiated stage 5 cells (FIG. 26B) showed no effect in response to insulin secretagogues in both the control and modified protocols.

In addition, the insulin and C-peptide content of the cells were determined. The results are shown in Table 2. C-peptide was produced from the differentiated cells indicating the ability of these cells to synthesise and process proinsulin. There was no significant difference in the C-peptide and insulin contents between differentiated stages 4 and 5 cells. However, supplementation with GIP(LysPAL¹⁶) resulted in a 1.5 and 1.3-fold increase in C-peptide content of cells at stages 4 and 5 cells, respectively, compared to control (P<0.001).

TABLE 2
Effects of various differentiation conditions on insulin
and C-peptide contents of differentiated D3 cluster cells

| | |
|---|---|
| Stage 4 | Stage 5 |

Insulin (pmol/mg protein/20 mins)

| | | |
|---|---|---|
| Control | 8311 ± 411.2 | 7370 ± 790.9 |
| GIP(LysPAL¹⁶) | 9154 ± 291.1 | 8532 ± 626.5 |

C-Peptide (pmol/mg protein/20 mins)

| | | |
|---|---|---|
| Control | 85.92 ± 5.44 | 94.03 ± 2.70 |
| GIP(LysPAL¹⁶) | 124.5 ± 3.13*** | 121.9 ± 3.43*** |

Overnight cultures were performed under various culture conditions. Values are the mean ± SEM for 8 separate observations.
***P < 0.001 compared with respective test reagent following control culture.

### Example 18

### Effects of N-AcGIP(PEG)

N-AcGIP(PEG) comprises a GIP molecule in which PEG is attached to the C-terminus and the Acetyl group was attached to the opposite end of the molecule-giving N-Ac-GIP(Peg).

This example shows the effects of N-AcGIP(PEG) administration on food intake and body weight of 12 weeks old ob/ob mice. The results are shown in FIG. 27. Daily injection of N-AcGIP(PEG) (25 nmoles/kg/day) did not affect body weight or food intake when administered daily over a 14 day period.

This example also shows the effects of N-AcGIP(PEG) administration on plasma glucose and insulin in 12 weeks old ob/ob mice. The results are shown in FIG. 28 which are a set of line graphs. Daily administration of N-AcGIP(PEG) (25 nmoles/kg/day) decreased non-fasting glucose without appreciable change in circulating insulin.

This example further shows the effects of N-AcGIP(PEG) administration on plasma glucose and insulin responses of 12 weeks old ob/ob mice to intraperitoneal glucose (18 mmol/kg body weight). The results are shown in FIG. 29, which are a set of line graphs with bar charts. 14 days administration of N-AcGIP(PEG) (25 mmoles/kg/day) improved glucose tolerance, lowered plasma glucose concentrations and reduced the overall glycaemic response after administration of intraperitoneal glucose load. Plasma insulin concentrations and the accompanying insulin response were enhanced by N-AcGIP(PEG) treatment, suggesting a beneficial effect on pancreatic beta cell function.

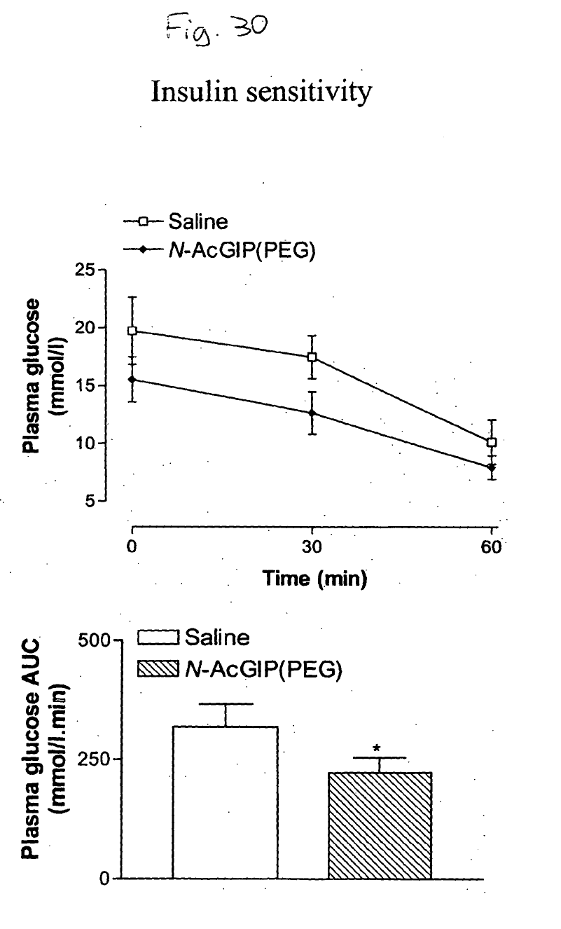

This example still further shows the effects of N-AcGIP(PEG) administration on insulin sensitivity in 12 weeks old ob/ob mice The results are shown in FIG. 30, which are a set of line graphs with bar charts. After 14 days administration of N-AcGIP(PEG), ob/ob mice displayed lower plasma glucose concentrations after administration of insulin. The overall glycaemic response was enhanced, consistent with reduction of insulin resistance.

While this invention has been particularly shown and described with references to preferred embodiments thereof, it will be understood by those skilled in the art that various changes in form and details may be made therein without departing from the scope of the invention encompassed by the appended claims.

1. A peptide analogue of GIP(1-42), wherein the peptide analogue is at least 12 amino acid residues from the N-terminal end of GIP(1-42) and wherein the peptide analogue comprises at least one amino acid substitution or modification, said at least one modification being fatty acid addition at an epsilon amino group of at least one lysine residue.

2. A peptide analogue as claimed in claim 1, wherein the at least one amino acid substitution comprises substitution of lysine for one or more of the residues and the at least one amino acid modification comprises fatty acid addition at an epsilon amino group of said at least one substituted lysine residue.

3. A peptide analogue as claimed in claim 1, wherein the peptide analogue further comprises an amino acid substitution of cysteine for one or more of the residues and wherein the modification is the addition of a polyethylene glycol (PEG) molecule at said at least one substituted cysteine residue.

4. A peptide analogue as claimed in claim 1, wherein the amino acid modification comprises the reaction of an acyl radical having a saturated or unsaturated, linear or branched aliphatic chain, of from 4 to 22 carbons, with an epsilon amino group of at least one lysine, or substituted lysine, residue.

5. A peptide analogue as claimed in claim 1, the peptide analogue being selected from GIP(LysPAL¹⁶), GIP(LysPAL³⁰), GIP(LysPAL³²), GIP(LysPAL³³) or GIP(LysPAL³⁷).

6. A peptide analogue as claimed in claim 5, the peptide analogue being selected from GIP(LysPAL¹⁶) or GIP(LysPAL³⁷).

7. A peptide analogue of GIP(1-42), wherein the peptide analogue is at least 12 amino acid residues from the N-terminal end of GIP(1-42) and wherein the peptide analogue comprises at least one amino acid substitution or modification, wherein said at least one modification is the addition of a polyethylene glycol (PEG) molecule.

8. A peptide analogue as claimed in claim 7, wherein said at least one modification is the addition of a polyethylene glycol (PEG) molecule at a position selected from the N-terminal position and the C-terminal position.

9. A peptide analogue as claimed in claim 7, wherein said at least one modification is the addition of a polyethylene glycol (PEG) molecule at a position other than a position selected from the N-terminal position and the C-terminal position.

10. A peptide analogue as claimed in claim 7, wherein the at least one amino acid substitution comprises the substitution of cysteine for one or more of the residues and wherein the at least one modification is the addition of a polyethylene glycol (PEG) molecule at said at least one substituted cysteine residue.

11. A peptide analogue as claimed in claim 7, wherein the at least one amino acid substitution comprises the substitution of lysine for one or more of the residues and the at least one amino acid modification by fatty acid addition at an epsilon amino group of said at least one substituted lysine residue.

12. A peptide analogue as claimed in claim 7, wherein the peptide analogue further comprises an amino acid modification at position 1.

13. A peptide analogue as claimed in claim 12, wherein the N-terminal amino acid modification is selected from N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule.

14. A peptide analogue as claimed in claim 7, wherein the base peptide is GIP(1-12), GIP(1-13), GIP(1-14), GIP(1-15), GIP(1-16), GIP(1-17), GIP(1-18), GIP(1-19), GIP(1-20), GIP(1-21), GIP(1-22), GIP(1-23), GIP(1-24), GIP(1-25), GIP(1-26), GIP(1-27), GIP(1-28), GIP(1-29), GIP(1-30), GIP(1-31), GIP(1-32), GIP(1-33), GIP(1-34), GIP(1-35), GIP(1-36), GIP(1-37), GIP(1-38), GIP(1-39), GIP(1-40), GIP(1-41), GIP(1-42); which base peptide possesses an amino acid modification with PEG at its C-terminal end.

15. A peptide analogue as claimed in claim 14, wherein the N-terminal modification is acylation.

16. A peptide analogue as claimed in claim 7, wherein the peptide analogue is selected from N-AcGIP(1-12)(PEG), N-AcGIP(1-13)(PEG), N-AcGIP(1-14)(PEG), N-AcGIP(1-15)(PEG), N-AcGIP(1-16)(PEG), N-AcGIP(1-17)(PEG), N-AcGIP(1-18)(PEG), N-AcGIP(1-19)(PEG), N-AcGIP(1-20)(PEG), N-AcGIP(1-21)(PEG), N-AcGIP(1-22)(PEG), N-AcGIP(1-23)(PEG), N-AcGIP(1-24)(PEG), N-AcGIP(1-25)(PEG), N-AcGIP(1-26)(PEG), N-AcGIP(1-27)(PEG), N-AcGIP(1-28)(PEG), N-AcGIP(1-29)(PEG), N-AcGIP(1-30)(PEG), N-AcGIP(1-31)(PEG), N-AcGIP(1-32)(PEG), N-AcGIP(1-33)(PEG), N-AcGIP(1-34)(PEG), N-AcGIP(1-35)(PEG), N-AcGIP(1-36)(PEG), N-AcGIP(1-37)(PEG), N-AcGIP(1-38)(PEG), N-AcGIP(1-39)(PEG), N-AcGIP(1-40)(PEG), N-AcGIP(1-41)(PEG), N-AcGIP(1-42)(PEG)

17. A peptide analogue as claimed in claim 1 or 7, wherein said at least one amino acid substitution or modification is at a position other than positions 1, 2 and 3.

18. A peptide analogue as claimed in claim 1 or 7, wherein said at least one amino acid substitution or modification is at one or more of positions 1, 2 and 3.

19. A peptide analogue as claimed in claim 1 or 7, wherein said at least one amino acid substitution is at one or both of positions 1, 2 and 3 or wherein said at least one amino acid modification is at one or both of positions 2 and 3.

20. A peptide analogue as claimed in claim 1 or 7, wherein said at least one amino acid substitution comprises an L-amino acid selected from L-alanine, L-arginine, L-asparagine, L-aspartic acid, L-cysteine, L-glycine, L-glutamic acid, L-glutamine, L-histidine, L-isoleucine, L-leucine, L-lysine, L-methionine, L-phenylalanine, L-proline, L-serine, L-threonine, L-tryptophan, L-tyrosine and L-valine or a D-amino acid selected from D-alanine, D-arginine, D-asparagine, D-aspartic acid, D-cysteine, D-glutamic acid, D-glutamine, D-glycine, D-histidine, D-isoleucine, D-leucine, D-lysine, D-methionine, D-phenylalanine, D-proline, D-serine, D-threonine, D-tryptophan, D-tyrosine and D-valine or by any other L- or D-amino acid other than those commonly encountered in the genetic code, including beta amino acids such as beta-alanine and omega amino acids such as 3-amino propionic, 4-amino butyric, etc, ornithine, citrulline, homoarginine, t-butyl alanine, t-butyl glycine, N-methyl isoleucine, phenylglycine, cyclohexylalanine, norleucine, cysteic acid, and methionine sulfoxide.

21. A pharmaceutical composition comprising a peptide analogue as claimed in claim 1 or 7, in association with a pharmaceutically acceptable carrier.

22. A pharmaceutical composition as claimed in claim 21, further comprising a therapeutically effective amount of an agent having an antidiabetic effect.

23. A method for ameliorating or restoring age related decreased pancreatic function, the method comprising administering a peptide analogue of GIP(1-42), wherein the peptide analogue is at least 12 amino acid residues from the N-terminal end of GIP(1-42) and wherein the peptide analogue comprises at least one amino acid substitution or modification.

24. The method as claimed in claim 23, wherein said at least one modification is at position 1, the modification being selected from N-terminal alkylation, N-terminal acetylation, N-terminal acylation, the addition of an N-terminal isopropyl group, the addition of an N-terminal pyroglutamic acid, or the addition of an N-terminal polyethylene glycol (PEG) molecule.

25. The method as claimed in claim 24, wherein the modification is N-terminal acetylation

26. The method as claimed in claim 23, wherein the peptide analogue is covalently attached to a polyethylene glycol (PEG) molecule.

27. The method as claimed in claim 23, wherein the peptide analogue comprises a modification by fatty acid addition at an epsilon amino group of at least one lysine residue.

28. The method as claimed in claim 27, wherein the modification is the linking of a C-8 octanoyl group, a C-10 decanoyl group, a C-12 lauroyl group, a C-14 myristoyl group, a C-16 palmitoyl group, a C-18 stearoyl group, or a C-20 acyl group to the epsilon amino group of a lysine residue.

29. The method as claimed in claim 28, where the lysine residue is selected from the group consisting of Lys¹⁶, Lys³⁰, Lys³², Lys³³ and Lys³⁷.

30. The method as claimed in claim 29, wherein the peptide analogue is N-AcGIP(LysPAL¹⁶), N-AcGIP(LysPAL³⁷), GIP(LysPAL¹⁶) or GIP(LysPAL³⁷).

31. The method as claimed in claim 23, wherein the medicament further comprises a pharmaceutically acceptable carrier.

32. The method as claimed in claim 23, wherein the peptide analogue is in the form of a pharmaceutically acceptable salt.

33. The method as claimed in claim 32, wherein the peptide analogue is in the form of a pharmaceutically acceptable acid addition salt.

34. The method as claimed in claim 23, wherein the pharmaceutical composition further comprises a therapeutically effective amount of an agent having an antidiabetic effect.

35. A peptide analogue as claimed in claim 4, wherein the amino acid modification comprises the reaction of an acyl radical selected from C-8 octanoyl, C-10 decanoyl, C-12 lauroyl, C-14 myristoyl, C-16 palmitoyl, C-18 stearoyl, or C-20 acyl with the epsilon amino group of a lysine, or substituted, residue.

36. The peptide analogue as claimed in claim 14, comprising a further amino acid modification at its N-terminal end.

37. The peptide analogue as claimed in claim 15, wherein the N-terminal modification is acetylation.

38. The peptide analogue as claimed in claim 16, wherein the peptide analogue is N-AcGIP(PEG).

39. The method of claim 23, wherein the, or each, said at least one amino acid substitution comprises an amino acid substitution of lysine or cysteine for one or more of the residues.

40. The method of claim 25, wherein the peptide analogue is N-Ac(GIP).
